(19) United States
(12) Patent Application Publication
(10) Pub. No.: US 2024/0226539 A1
(43) Pub. Date: Jul. 11, 2024

(54) MONITORING STIMULATING ASSEMBLY INSERTION

(71) Applicant: Cochlear Limited, Macquarie University (AU)

(72) Inventors: John Michael Heasman, Hampton (AU); Peter Gibson, South Coogee (AU); Stephen O'Leary, Melbourne (AU); Luke Campbell, Melbourne (AU)

(21) Appl. No.: 18/613,468

(22) Filed: Mar. 22, 2024

Related U.S. Application Data
(63) Continuation-in-part of application No. 17/684,768, filed on Mar. 2, 2022.
Application No. 17/684,768 is a continuation of application No. 16/703,926, filed on Dec. 5, 2019, now Pat. No. 11,305,109.
Application No. 16/703,926 is a continuation of application No. 15/245,547, filed on Aug. 24, 2016, now Pat. No. 10,500,392.

(51) Int. Cl. A61N 1/05; A61B 5/00; A61B 5/12; A61B 5/38; A61N 1/08; A61N 1/36

(52) U.S. Cl. CPC A61N 1/0541; A61B 5/125; A61B 5/38; A61B 5/4851; A61B 5/686; A61N 1/08; A61N 1/36036

(57) ABSTRACT

Presented herein are techniques for monitoring the insertion of an intra-cochlear stimulating assembly for the occurrence of one or more insertion stop conditions. The insertion stop conditions are detectable events indicating that movement of the stimulating assembly into a recipient's cochlea should be at least temporarily stopped. The insertion monitoring is based on objectively measured inner ear potentials, such as acoustically-evoked potentials.

### CROSS-REFERENCE TO RELATED APPLICATIONS

This application is a continuation-in-part application of U.S. patent application Ser. No. 17/684,768, filed on Mar. 2, 2022, which is a continuation application of U.S. patent application Ser. No. 16/703,926, filed on Dec. 5, 2019, which is a continuation application of U.S. patent application Ser. No. 15/245,547, entitled “Monitoring Stimulating Assembly Insertion,” filed on Aug. 24, 2016, the content of which is hereby incorporated by reference herein.

### BACKGROUND

### Field of the Invention

The present invention relates generally to insertion of implantable stimulating assemblies.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

### SUMMARY

In one aspect, a method is provided. The method comprises: determining that an insertion stop condition for an electrode array of a cochlear implant has occurred during implantation of the electrode array in a recipient, wherein the insertion stop condition is based on an objective measure of the recipient's residual hearing in the cochlea and a subjective measure of the recipient's residual hearing in the cochlea.

In another aspect, a method is provided. The method comprises: during insertion of an electrode array into a cochlea of a recipient, monitoring signals indicative of residual hearing of the cochlea of the recipient; detecting, based on the signals indicative of residual hearing, a target stop condition associated with a predetermined target stop point, wherein the target stop condition is based on an objective measure of the recipient's residual hearing in the cochlea and a subjective measure of the recipient's residual hearing in the cochlea; and responsive to detection of the target stop condition, initiating a feedback mechanism indicating that an electrode of the electrode array has been inserted to a target insertion depth and to stop insertion of the electrode array into the cochlea.

### BRIEF DESCRIPTION OF THE DRAWINGS

Embodiments of the present invention are described herein in conjunction with the accompanying drawings, in which:

FIG. 1A is a schematic diagram illustrating a cochlear implant system implanted in a head of a recipient and an intra-operative insertion monitoring system for use during implantation of the cochlear implant, in accordance with embodiments presented herein;

FIG. 1B is a side view of a recipient wearing a sound processing unit of the cochlear implant system of FIG. 1A;

FIG. 1C is a schematic view of components of the cochlear implant system of FIG. 1A;

FIG. 1D is a block diagram illustrating further details of the cochlear implant system and the intra-operative system of FIG. 1A with which aspects of the techniques presented herein can be implemented;

FIG. 1E is a block diagram of the intra-operative insertion monitoring system of FIGS. 1A and 1D with which aspects of the techniques presented herein can be implemented;

FIG. 2A is a diagram schematically illustrating a target stop point, in accordance with embodiments presented herein;

FIG. 2B is a graph illustrating measured cochlear microphonic amplitudes relative to time, in accordance with embodiments presented herein;

FIGS. 2C-2H are a series of images illustrating different stages of insertion of an electrode array into a plastic model of a cochlea, in accordance with embodiments presented herein;

FIG. 3A is a flowchart of an example method in accordance with embodiments presented herein;

FIG. 3B is a flowchart of an example method in accordance with embodiments presented herein;

FIG. 3C is a flowchart of an example method in accordance with embodiments presented herein;

FIGS. 4A, 4B, and 4C are schematic diagrams illustrating various example implementations of the method of FIG. 3C involving subjective feedback, in accordance with embodiments presented herein;

FIGS. 5A and 5B are diagrams illustrating cochlear microphonic amplitudes measured during an atraumatic insertion;

FIGS. 6A and 6B are diagrams illustrating cochlear microphonic amplitudes measured during a traumatic insertion;

FIG. 7A is a flowchart of another example method in accordance with embodiments presented herein;

FIG. 7B is a flowchart of another example method in accordance with embodiments presented herein;

FIG. 7C is a flowchart of another example method in accordance with embodiments presented herein; and

FIGS. 8A, 8B, and 8C schematically illustrate the effect of over-insertion and pull back of a stimulating assembly that can be initiated using the techniques presented herein.

### DETAILED DESCRIPTION

Auditory/hearing device users/recipients (collectively and generally referred to herein as “recipients”) suffer from different types of hearing loss (e.g., conductive and/or sensorineural) and/or different degrees/severity of hearing loss. However, it is now common for many hearing device recipients to retain some residual acoustic hearing ability (residual hearing), which in turn can be capitalized upon by hearing devices through the use of acoustic stimulation (e.g., natural or amplified acoustic stimulation signals) or mechanical stimulation (e.g., mechanical stimulation signals) in combination with electrical stimulation (i.e., electrical stimulation signals). Even in the absence of residual hearing, it can be beneficial to match the electrical stimulus delivered by an electrically stimulating hearing device (e.g., a cochlear implant) to the natural tonotopy of the cochlea. For example, mapping electrical stimulation to the tonotopy of the implanted ear can help improve hearing performance in recipient with single sided deafness because the hearing perception created by the hearing device better correlates with what the recipient hears with their contralateral ear. For ease of description, the terms “acoustic stimulation” or “acoustic stimulation signals” are used herein to refer to the delivery of unaided acoustic stimulation (natural hearing), the delivery of amplified acoustic stimulation signals, as well as the delivery of mechanical stimulation signals, since all of these mechanisms result in motion of the cochlea fluid.

In certain embodiments, acoustic stimulation can be delivered contemporaneously (e.g., simultaneously) with electrical stimulation to the same ear of a recipient. Typically, due to the mechanics of hearing loss, the acoustic stimulation is used to present sound signal components corresponding to the lower frequencies of input sound signals (as determined from the residual hearing capabilities of the implanted ear), while the electrical stimulation is used to present sound signal components corresponding to the higher frequencies. The tonotopic region of the cochlea where the sound or stimulation output transitions from acoustic stimulation to electric stimulation is called the cross-over frequency region.

The combination of the acoustic stimulation with electrical stimulation (combined acoustic-electric hearing) is advantageous because the acoustic or mechanical stimulation adds a more “natural” sound to their hearing perception relative to delivery of only electrical stimulation signals. The addition of the acoustic stimulation can, in some cases, also provide improved pitch and music perception and/or appreciation, as the acoustic signals can contain a more salient lower frequency (e.g., fundamental pitch, F0) representation than is possible with electrical stimulation. Other benefits can include, for example, improved sound localization, binaural release from unmasking, an increased ability to distinguish sounds in a noisy environment, etc.

Due to the benefits of combined acoustic-electric hearing, there is a desire to preserve as much of the recipient's residual hearing as possible during implantation of the stimulating assembly into a recipient's cochlea and/or ensure adequate alignment of the stimulating assembly with the natural tonotopy of the cochlea. Progressive improvements in the design of intra-cochlear stimulating assemblies (electrode arrays), surgical implantation techniques, tooling, etc. have enabled atraumatic surgeries which preserve at least some of the recipient's fine inner ear structures (e.g., cochlea hair cells) and the natural cochlea function, particularly in the lower frequency regions of the cochlea. With these improvements, clinical outcomes for recipients have improved significantly. Nonetheless, many factors can compound the probability of a good surgical outcome, including surgical skill and experience of the surgeon(s), cochlear anatomy, stimulating assembly type, etc. As a result, there remain cases in which the stimulating assembly insertion and associated surgical practices result in significant trauma to the cochlea and/or improper placement of the stimulating assembly, thereby resulting in suboptimal clinical benefit.

Presented herein are techniques for objectively and/or subjectively monitoring the insertion of an intra-cochlear stimulating assembly for the occurrence of one or more “insertion stop conditions.” The insertion stop conditions are events indicating that insertion of the stimulating assembly into a recipient's cochlea should be at least temporarily stopped/ceased. In certain embodiments, the insertion monitoring is based on objectively measured inner ear responses/potentials, such as acoustically-evoked potentials. For example, in one form, the measured inner ear potentials are used to determine when a stimulating assembly has been appropriately aligned with the tonotopy of the cochlea and/or inserted to a pre-selected/programmed insertion depth that is derived from one or more pre-operative measurements, such as an audiogram. In another form, the measured inner ear potentials are used to determine if the stimulating assembly contacts an internal structure of the cochlea, such as the basilar membrane, during the insertion procedure (e.g., at the distal end of the stimulating assembly or along the length of the stimulating assembly). The inner ear potentials captured during insertion of the stimulating assembly can be stored (e.g., in non-volatile memory associated with a surgical guidance system), and subsequently used to program the hearing device for the recipient. For example, a fitting system can derive a personalized frequency allocation table from the measured inner ear potentials that adjusts the frequency range assigned to each electrode contact on the stimulating assembly based on the position of the stimulating assembly within the cochlea post-insertion (i.e., matching the frequency allocated to the electrodes of the stimulating assembly to the tonotopy of the cochlea).

As described further below, a pre-selected insertion depth for a stimulating assembly, which is defined in terms of a frequency corresponding to residual hearing of the recipient and/or a frequency that represents proper alignment of the stimulating assembly with the natural tonotopy of the implanted ear, is referred to herein as a “target stop point.” In other words, a target stop point is a pre-operatively defined insertion stop frequency. A “target stop condition” occurs when the stimulating assembly is inserted into the cochlea such that a portion of the stimulating assembly (e.g., a distal end of the stimulating assembly and/or one or more stimulating contacts) is located at a tonotopic region corresponding to the pre-operatively defined insertion stop frequency. Contact with an internal structure of the cochlea that is likely to impeded residual hearing, such as impingement on the basilar membrane, is referred to herein as an “error stop point” so as to trigger an “error stop condition.”

In accordance with the techniques presented herein, detection of one of these two types of “stop points” or “stop conditions” triggers a feedback mechanism to stop or alter the insertion process. For example, the feedback mechanism can operate to halt automated insertion of a stimulating assembly (e.g., in a robotic or automated insertion) and/or to generate a notification to a surgeon (e.g., a notification to halt insertion of the stimulating assembly). In general, the error stop condition operates as a safety mechanism that is able to detect adverse events that occur during the insertion process, while the target stop condition provides the surgeon with evidence-based information that is recipient-centric (personalized) and standardized across recipient populations.

Also as described below, in addition to the stop conditions, embodiments of the present invention can also detect one or more “insertion warning conditions.” An insertion warning condition occurs when the system determines that insertion of the stimulating assembly is approaching, or is likely to soon satisfy, one of the insertion stop conditions. The detection of an insertion warning condition can result in the initiation of a feedback mechanism indicating that insertion of the stimulating assembly into the cochlea should be slowed (e.g., to slow automated forward movement of a stimulating assembly by a surgical robot, an alert or notification to a surgeon to slow forward movement, modify insertion to prevent motion of the stimulating assembly outwards towards the scala wall, etc.).

As described further below, in addition to objective measurements (e.g., ECochG recordings, etc.), embodiments of the present invention can also detect one or more insertion stop conditions or insertion warning conditions based on “subjective feedback” obtained from the recipient during insertion. In these embodiments, the electrical stimulation and/or acoustic stimulation is delivered to the recipient (e.g., either in successive bursts in the same ear, or simultaneously or sequentially in opposite ears), and the recipient is asked to compare the two stimuli and indicate whether they match or not.

For ease of illustration, embodiments are primarily described herein with reference to insertion of stimulating assemblies forming part of a cochlear implant. However, it is to be appreciated that the techniques presented herein can also be partially or fully implemented by any of a number of different types of devices, including hearing devices, implantable medical devices, etc. having a stimulation assembly configured to be inserted into the body of a recipient. As used herein, the term “hearing device” is to be broadly construed as any device that delivers sound signals to a user in any form, including in the form of acoustic stimulation, mechanical stimulation, electrical stimulation, etc. As such, a hearing device can be a device for use by a hearing-impaired person (e.g., hearing aids, middle ear auditory prostheses, bone conduction devices, direct acoustic stimulators, electro-acoustic hearing prostheses, auditory brainstem stimulators, bimodal hearing prostheses, bilateral hearing prostheses, dedicated tinnitus therapy devices, tinnitus therapy device systems, combinations or variations thereof, etc.) or a device for use by a person with normal hearing (e.g., consumer devices that provide audio streaming, consumer headphones, earphones and other listening devices). In other examples, the techniques presented herein can be implemented by, or used in conjunction with, various implantable medical devices, such as vestibular devices, visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

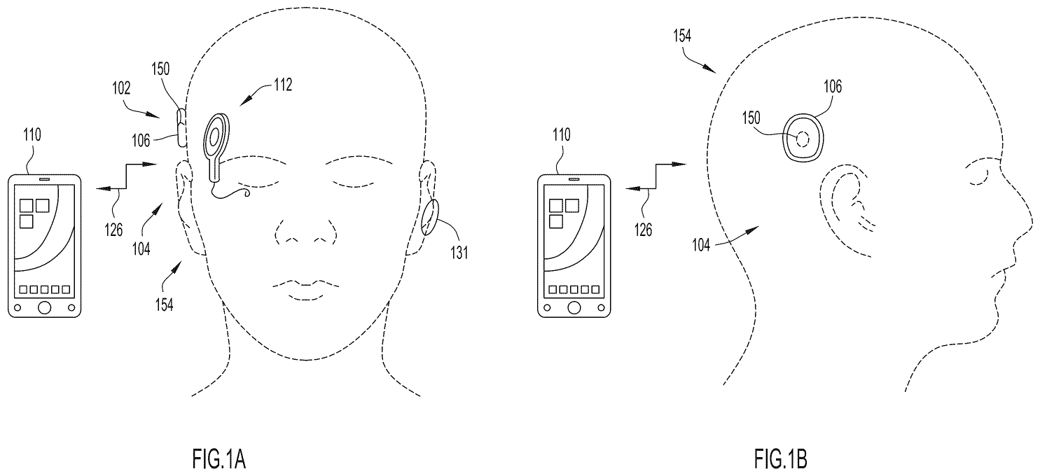

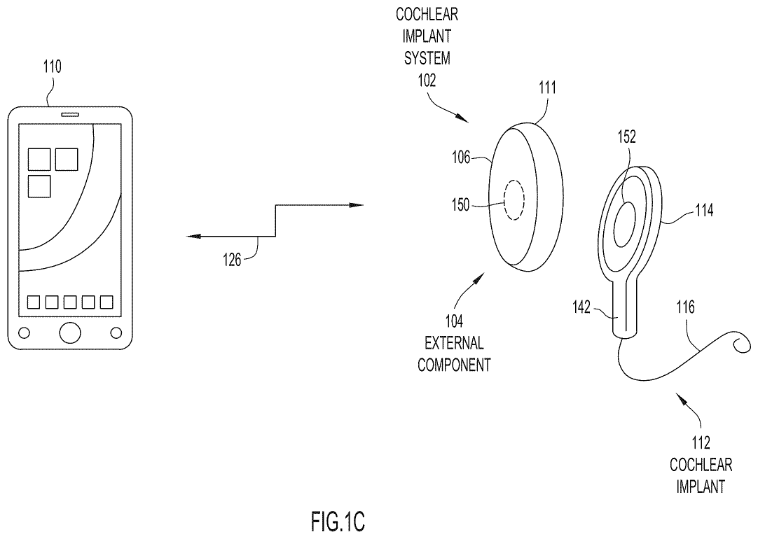

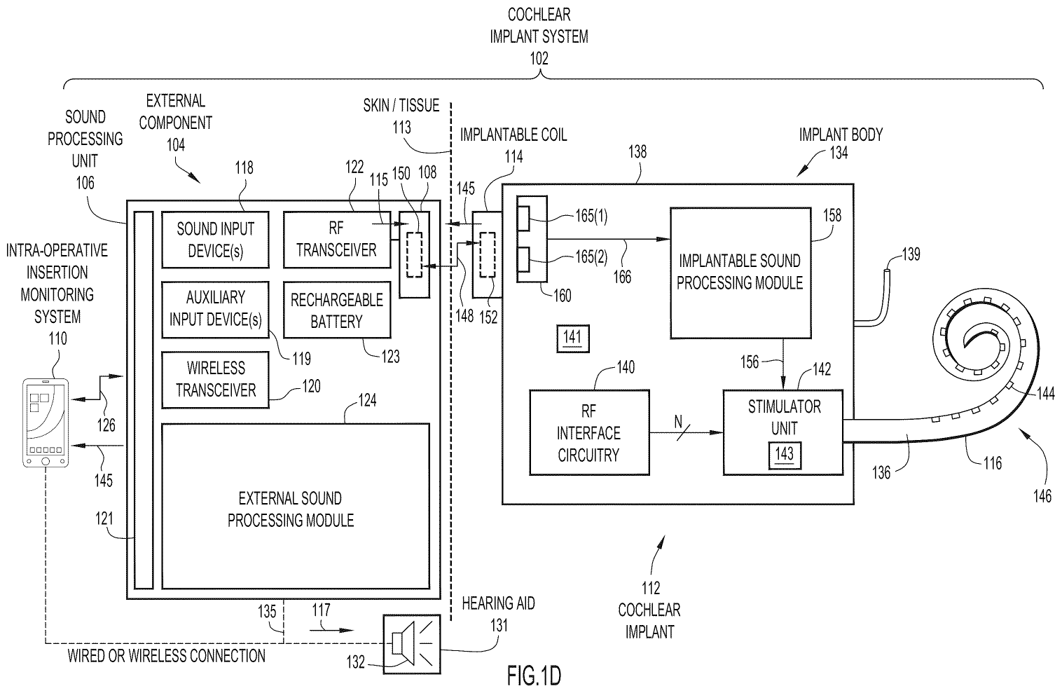

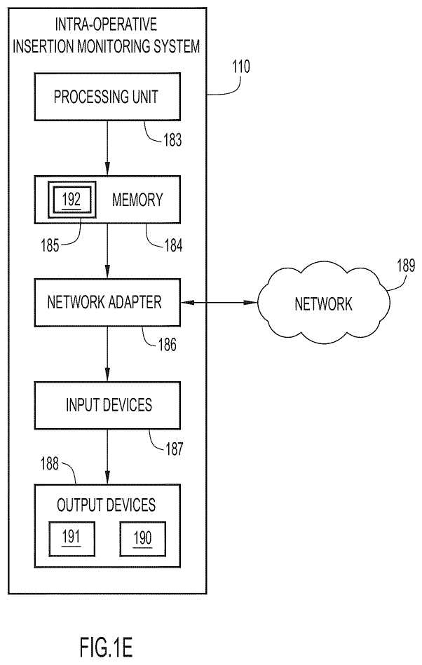

FIGS. 1A-1E are diagrams of an illustrative intra-operative insertion monitoring system 110 (also referred to herein as intra-operative system 110) configured to implement the techniques presented herein during implantation of implantable medical devices. To facilitate understanding of the techniques presented herein, the intra-operative system 110 is shown with an example cochlear implant system 102 in FIGS. 1A, 1C, and 1D. The illustrative intra-operative system 110, which is shown in greater detail in FIG. 1E, can be, for example, a computing device, such as a remote assistant for the cochlear implant system (e.g., a remote control unit), a computer (e.g., laptop, desktop), a hand-held device (e.g., tablet), a mobile device (e.g., a smartphone), etc., or other device configured for communication with the cochlear implant system 102. The intra-operative system 110 and the cochlear implant system 102 (e.g., a sound processing unit 106 and/or a cochlear implant 112) are configured to wirelessly communicate via a bi-directional communication link 126 (e.g., a short-range communication link, a near-field communication link (NFC), a Bluetooth link, a Bluetooth Low Energy (BLE) link, a proprietary link, etc.). In certain embodiments, the intra-operative system 110 can include both a computing device and an external component of a cochlear implant system (e.g., a sound processing unit). In some embodiments, the external component that provides a communication interface between the implantable component of the cochlear implant system 102 (e.g., a cochlear implant) can be a dedicated surgical processor. Before describing details of the intra-operative system 110 and the insertion monitoring techniques presented herein, a description of the cochlear implant system 102 is first provided below for context.

FIGS. 1A-1D illustrate an example cochlear implant system 102 with which aspects of the techniques presented herein can be implemented. As shown in FIGS. 1A-1D, the cochlear implant system 102 includes an external component 104 and an internal/implantable component 112. The external component 104 is configured to be directly or indirectly attached to the body of a recipient, while the implantable component 112 is configured to be subcutaneously implanted within the head of the recipient (i.e., under the skin/tissue of the recipient). In the examples of FIGS. 1A-1D, the implantable component 112 is sometimes referred to as a “cochlear implant.” The cochlear implant system 102 operates with an intra-operative insertion monitoring system 110.

FIG. 1A is a schematic diagram that illustrates the cochlear implant 112 implanted in the head 154 of a user, while FIG. 1B is a schematic diagram illustrating the external component 104 worn on the head 154 of the user. FIG. 1C is another schematic view of the cochlear implant system 102, while FIG. 1D illustrates further details of the cochlear implant system 102 and the intra-operative insertion monitoring system 110, in accordance with example embodiments presented herein. For ease of description, FIGS. 1A-1D will generally be described together.

In the examples of FIGS. 1A-1D, the external component 104 comprises a sound processing unit 106, an external coil 108, and, generally, a magnet fixed relative to the external coil 108. The cochlear implant 112 includes an implantable coil 114, an implant body 134, and an elongate stimulating assembly 116 configured to be implanted in the user's cochlea. In one example, the sound processing unit 106 is an off-the-ear (OTE) sound processing unit, sometimes referred to herein as an “OTE component,” that is configured to send data and power to the implantable component 112 as described below. In general, an OTE sound processing unit is a component having a generally cylindrically shaped housing 111 and which is configured to be magnetically coupled to the user's head 154. For example, the sound processing unit 106 also includes an integrated external magnet 150 configured to be magnetically coupled to an internal/implantable magnet 152 in the implantable component 112. The OTE sound processing unit 106 also includes an integrated external (headpiece) coil 108 (the external coil 108) that is configured to be inductively coupled to the implantable coil 114 of the implantable component 112 as described below.

It is to be appreciated that the OTE sound processing unit 106 is merely illustrative of the external devices that could operate with implantable component 112. For example, in alternative examples, the external component 104 can comprise a behind-the-ear (BTE) sound processing unit configured to be attached to, and worn adjacent to, the recipient's ear. In general, a BTE sound processing unit comprises a housing that is shaped to be worn on the outer ear of the user and is connected to the separate external coil assembly via a cable, where the external coil assembly is configured to be magnetically and inductively coupled to the implantable coil 114. It is also to be appreciated that alternative external components could be located in the user's ear canal, worn on the body, etc.

The sound processing unit 106 comprises one or more input elements configured to capture and/or receive input signals (e.g., sound or data signals) at the sound processing unit 106. The one or more input elements include, for example, one or more sound input devices 118 (e.g., one or more external microphones, audio input ports, cable ports, telecoils, a wireless transceiver, etc.), one or more auxiliary input devices 119 (e.g., audio ports such as Direct Audio Input (DAI), data ports such as a Universal Serial Bus (USB) port, cable port, etc.), and a short-range wireless transmitter/receiver (wireless transceiver) 120 (e.g., for communication with intra-operative system 110), a charging coil 121, a closely-coupled radio frequency transmitter/receiver (RF transceiver) 122, at least one rechargeable battery 123, and an external sound processing module 124, each located in, on, or near the sound processing unit 106. The external sound processing module 124 can be configured to perform a number of operations, and can be formed by one or more processors (e.g., one or more digital signal processors (DSPs), one or more microprocessors, one or more uC cores, one or more microcontrollers, etc.), a memory device (memory), firmware, software, etc. arranged to perform operations described herein. That is, the external sound processing module 124 can be implemented as firmware elements, partially or fully implemented with digital logic gates in one or more application-specific integrated circuits (ASICs), partially or fully in software, etc.

As noted, the sound processing unit 106 can be, for example, an off-the-ear (OTE) sound processing unit, a behind-the-ear (BTE) sound processing unit, a body-worn sound processing unit, a button sound processing unit, etc. More generally, the OTE sound processing unit 106 is used for communication between the intra-operative insertion monitoring system 110 and the cochlear implant 112. As such, during a surgical procedure, the OTE sound processing unit 106 could be replaced by any other device that is able to communicate with the intra-operative insertion monitoring system 110 and the cochlear implant 112. In certain embodiment, the OTE sound processing unit 106 could be a so-called “surgical processor” having fewer capabilities than the OTE sound processing unit 106 (e.g., no sound processing logic, etc.). In various embodiments, the communication between the intra-operative insertion monitoring system 110 and the OTE sound processing unit 106 (or another device operating in place of the OTE sound processing unit 106, such as a surgical processor with reduced functionality), could communicate via a wireless or wired connection.

In addition, while FIGS. 1A-1D illustrate an arrangement in which the cochlear implant system 102 includes an external component, it is to be appreciated that embodiments of the present invention can be implemented in cochlear implant systems having alternative arrangements. For example, embodiments presented herein can be implemented with a totally implantable cochlear implant or other totally implantable medical device. A totally implantable medical device is a device in which all components of the device are configured to be implanted under skin/tissue of a recipient. Because all components are implantable, a totally implantable medical device operates, for at least a finite period of time, without the need of an external device/component. However, an external component can be used to, for example, charge the internal power source (battery) of the totally implantable medical device. Returning to the specific example of FIG. 1D, connected to at least one of the sound processing unit 106 or the intra-operative system 110 (e.g., via a cable 135) is an acoustic stimulator 131, such as a hearing aid component, bone conduction device, etc. In certain embodiments, the acoustic stimulator 131 can be communicatively coupled with the sound processing unit 106 or with the intra-operative system 110 via a wireless connection (i.e., rather than a wired connection via cable 135).

In one example, the acoustic stimulator 131 is an air conduction device (e.g., hearing aid component) that includes a receiver 132 (FIG. 1D) that can be, for example, positioned in or near the recipient's outer ear. The receiver 132 is an acoustic transducer (e.g., a speaker) that is configured to deliver acoustic signals (acoustic stimulation signals) to the recipient via the recipient's ear canal and middle ear. In other examples, the acoustic stimulator 131 is a bone conduction device. In such embodiments, the bone conduction device would send a signal to both ears, whereas the air conduction device sends air conduction signal to the single ear (the ear it is on). In certain examples, the acoustic stimulator 131 could generate a combination of air and bone conduction stimuli (e.g., include both an air conduction device and a bone conduction device). Such embodiments could, for example, enable a comparison with each ear separately with the bone conduction which is essentially binaural. (e.g., to compare each ear with the binaural acoustic condition and the electrical stimulus). In addition, different frequencies could be delivered the air conduction device and the bone conduction device.

As shown in FIG. 1D, the implantable component 112 comprises an implant body (main module) 134, a lead region 136, and an elongate intra-cochlear stimulating assembly 116, all configured to be implanted under the skin/tissue 113 of the recipient. The magnets 150 and 152 magnetically couple the external component 104 to the implantable component 112 through the skin/tissue 113. The implant body 134 generally comprises a hermetically-sealed housing 138 in which an internal transceiver unit, also referred to herein as etc. interface circuitry 140, at least one power source 141 (e.g., one or more batteries, one or more super-capacitors, etc.), and a stimulator unit 142 are disposed. The implant body 134 also includes an internal/implantable coil 114 that is generally external to the housing 138, but which is connected to the RF interface circuitry 140 (transceiver) via a hermetic feedthrough (not shown in FIG. 1D). Implantable coil 114 is typically a wire antenna coil comprised of multiple turns of electrically insulated single-strand or multi-strand platinum or gold wire. The electrical insulation of implantable coil 114 is provided by a flexible molding (e.g., silicone molding), which is not shown in FIG. 1D. Generally, a magnet 152 is fixed relative to the implantable coil 114.

The elongate stimulating assembly 116 is configured to be at least partially implanted in the recipient's cochlea, and includes a plurality of longitudinally spaced intra-cochlear electrical stimulating contacts (electrodes) 144 that collectively form a contact array (electrode array) 146 for delivery of electrical stimulation (current) to the recipient's cochlea. The stimulating assembly 116, which has a distal end and a proximal end, extends through an opening in the recipient's cochlea (e.g., cochleostomy, the round window, etc.). The proximal end is connected to stimulator unit 142 via lead region 136 and a hermetic feedthrough (not shown in FIG. 1D). Lead region 136 includes a plurality of conductors (wires) that electrically couple the electrodes 144 to the stimulator unit 142. The implantable component 112 also includes an electrode outside of the cochlea, sometimes referred to as an extra-cochlear electrode (ECE) 139.

Returning to the external component 104 (sound processing unit 106), the sound input devices 118 are configured to detect/receive input sound signals and to generate electrical input signals therefrom. The sound processing unit 106 is configured execute sound processing and coding (via the external sound processing module 124) to convert the electrical input signals received from the sound input devices 118 into output signals that represent acoustic and/or electric (current) stimulation for delivery to the recipient. That is, as noted, the cochlear implant system 102 operates to evoke perception by the recipient of sound signals received by the sound input devices 118 through the delivery of one or both of electrical stimulation signals and acoustic stimulation signals to the recipient. As such, depending on a variety of factors, the sound processing unit 106 is configured to convert the electrical input signals received from the sound input elements into a first set of output signals representative of electrical stimulation and/or into a second set of output signals representative of acoustic stimulation. The output signals representative of electrical stimulation are represented in FIG. 1D by arrow 115, while the output signals representative of acoustic stimulation are represented in FIG. 1D by arrow 117.

The output signals 115 are, in the examples of FIGS. 1A-1D, encoded data signals that are sent to the implantable component 112 via external coil 108. More specifically, the magnet 150 fixed relative to the external coil 108 and the magnet 152 fixed relative to the implantable coil 114 can facilitate the operational alignment of the external coil 108 with the implantable coil 114. This operational alignment of the coils enables the external coil 108 to transmit the encoded data signals, as well as power signals received from the rechargeable battery 123, to the implantable coil 114 via a closely-coupled wireless link formed between the external coil 108 and the implantable coil 114. In certain examples, external coil 108 transmits the signals to implantable coil 114 via a radio frequency (RF) link. However, various other types of energy transfer, such as infrared (IR), electromagnetic, capacitive and inductive transfer, can be used to transfer the power and/or data from an external component to an implantable component and, as such, FIG. 1D illustrates only one example arrangement. For example, the external coil 108 can be in electrical communication with a power supply (e.g., the rechargeable battery 123) and can induce a current in the implantable coil 114, via an inductive link between the coils 108 and 114, to supply power to the implantable component 112.

In general, the encoded data and power signals are received at the RF interface circuitry (transceiver) 140 and are provided to the stimulator unit 142. The stimulator unit 142 is configured to utilize the encoded data signals to generate electrical stimulation signals (e.g., current signals) for delivery to the recipient's cochlea via one or more stimulating contacts 144. In this way, the cochlear implant system 102 electrically stimulates the recipient's auditory nerve cells, bypassing absent or defective hair cells that normally transduce acoustic vibrations into neural activity, in a manner that causes the recipient to perceive one or more components of the received sound signals.

In a normal or fully functional ear, an acoustic pressure or sound wave (i.e., a sound signal) is collected by the outer ear and channeled into and through the ear canal. Disposed across the distal end of ear cannel is a tympanic membrane that vibrates in response to sound wave. This vibration is coupled to the oval window through three bones of middle ear. The middle ear bones serve to filter and amplify sound wave, causing the oval window to articulate, or vibrate, in response to vibration of tympanic membrane. This vibration sets up waves of fluid motion of the perilymph within the cochlea to active the cochlea hair cells. Activation of the hair cells causes appropriate nerve impulses to be generated and transferred through the recipient's spiral ganglion cells and auditory nerve to the brain where they are perceived as sound.

As noted above, it is common for cochlear implant recipients to retain at least part of this normal hearing functionality (e.g., residual hearing). Therefore, the cochlea of a cochlear implant recipient can be acoustically stimulated upon delivery of a sound signal to the recipient's outer ear without the aid of the cochlear implant 112 itself. In certain recipients, the normal hearing functionality can be enhanced through the use of an acoustic transducer in or near the outer ear and/or ear canal. In such recipients, the acoustic transducer is used to filter, enhance, and/or amplify a sound signal which is delivered to the cochlea via the middle ear bones and oval window, thereby creating waves of fluid motion of the perilymph within the cochlea. In some instances, normal hearing functionality can be objectively measured (e.g., inner ear and/or neural potentials) to an extent in recipients that do not possess useful residual hearing. As such, an ECochG recording used in accordance with embodiments presented herein can be initiated by the intra-operative insertion monitoring system 110. The ECochG recording involves the delivery of acoustic stimuli to the recipient's cochlea, and recording one or more responses of the cochlea to the acoustic stimulation. As used herein, the term acoustic stimuli refers to any type of stimulation that is delivered in a manner so as to set up waves of fluid motion of the perilymph within the cochlea that, in turn, activates the hair cells inside of the cochlea. As such, acoustic stimuli for performance of an ECochG recording in accordance with embodiments presented herein can be delivered via a recipient's normal hearing functionality, via an acoustic transducer, for example.

In the specific example of FIG. 1D, the receiver 132 of the hearing aid 131 is an acoustic transducer in the form of a speaker that is used to provide the acoustic stimulation to the cochlea of the recipient. That is, the receiver 132 is configured to utilize the output signals 117 to generate acoustic stimulation signals that are provided to the recipient's cochlea via the middle ear bones and oval window, thereby creating waves of fluid motion of the perilymph within the cochlea. FIG. 1D also illustrates that the stimulator unit 142 of the cochlear implant 112 includes one or more integrated amplifiers 143 configured to digitally record ECochG signals/responses that are induced in the cochlea by the acoustic stimulus. The RF interface circuitry 140 and the RF transceiver 122 cooperate to provide ECochG signal data (e.g., the captured ECochG signals and data associated with captured ECochG signals, including recording site and time information) to the sound processing unit 106 (or surgical processor), where the ECochG signal data is then provided to the intra-operative insertion monitoring system 110. The ECochG signal data is generally represented in FIG. 1D by arrow 145. As noted, presented herein are several options for recording and analyzing ECochG signals during insertion of the stimulating assembly into the cochlea, as described below in greater detail.

Although FIG. 1D illustrates the use of a receiver to deliver acoustic stimulation to the recipient, it is to be appreciated that other types of devices can be used in other embodiments, such as bone conduction devices as described above. It is also to be appreciated that embodiments of the present invention can be implemented in other hearing prostheses, implantable medical devices, stimulation devices, and other arrangements than that shown in FIGS. 1A-1D. For example, it is to be appreciated that embodiments of the present invention can be implemented with fully-implantable hearing prostheses in which the sound processor, power supply, etc. are all implanted within a recipient so that the hearing prosthesis can operate, for at least a period of time, without the presence of an external component. It is also to be appreciated that the embodiments of the present invention can be implemented with implantable hearing prostheses that do not deliver acoustic stimulation to a recipient (e.g., cochlear implants, auditory brainstem stimulators, etc.).

As noted, FIG. 1E is block diagram illustrating one example arrangement for an intra-operative insertion monitoring system 110, configured to perform operations in accordance with embodiments presented herein. As noted above, illustrative intra-operative system 110 can be, for example, a computing device, such as a remote assistant for the cochlear implant system, a personal computer (e.g., laptop, desktop), a hand-held device (e.g., tablet), a mobile device (e.g., smartphone), a surgical system, etc., or other electronic device configured for communication with an implantable medical device system, such as cochlear implant system 102 of FIGS. 1A-1D, and having the capabilities to perform the associated operations described elsewhere herein. In certain embodiments, the intra-operative system 110 can include both a computing device and an external component of a cochlear implant.

Referring specifically to the arrangement of FIG. 1E, the intra-operative insertion monitoring system 110 comprises At least one processing unit 183 and a memory device (memory) 184. The processing unit 183 includes one or more hardware and/or software processors (e.g., central processing units (CPUs), microprocessors, control circuitry, etc.) that can obtain and execute instructions. The processing unit 183 can communicate with and control the performance of other components of the intra-operative system 110.

The memory 184 is one or more software and/or hardware-based computer-readable storage media operable to store information accessible by the processing unit 183. The memory 184 can store, among other things, instructions executable by the processing unit 183 to implement applications or cause performance of operations described herein, as well as other data. The memory 184 can comprise any one or more of one or more tangible (non-transitory) computer readable storage media, non-volatile memory such as read-only memory (ROM), volatile memory such as random-access memory (RAM), or a combination of RAM and ROM. In other examples, the memory 184 can comprise any one or more of electronically-erasable programmable read-only memory (EEPROM), flash memory devices, magnetic disk storage media devices, optical storage media devices, solid state storage devices, or other physical/tangible memory storage devices usable to store information and instructions for later access. By way of example and not limitation, the memory 184 can include wired media, such as a wired network or direct-wired connection, and wireless media, such as acoustic, RF, infrared, cellular, or combinations thereof. In certain embodiments, the memory 184 comprises (stores) intra-operative logic 185 that, when executed, enables the processing unit 183 to perform aspects of the techniques presented herein.

In the illustrated example of FIG. 1E, the intra-operative system 110 further comprises a network adapter 186 (e.g., one or more wireless communication interfaces that enables communication with the external component 104 and/or the cochlear implant 112), one or more input devices 187, and one or more output devices 188. The intra-operative system 110 can also include other components, such as a system bus, component interfaces, a graphics system, a power source (e.g., a battery), etc. The network adapter 186 is a component that provides network access (e.g., access to at least one network 189). The network adapter 186 can provide wired or wireless network access and can support one or more of a variety of communication technologies and protocols, such as Ethernet, cellular, Bluetooth, near-field communication (NFC), and radio frequency (RF) communication, among others. The network adapter 186 can include one or more antennas and associated components configured for wireless communication according to one or more wireless communication technologies and protocols.

In certain examples, the one or more communications interfaces of the network adapter 186 comprise one or more elements for wired or wireless communication with a hearing prosthesis. The communications interfaces of the network adapter 186 can comprise, for example, a short-range wireless transceiver, such as a Bluetooth® transceiver that communicates using short-wavelength Ultra High Frequency (UHF) radio waves in the industrial, scientific and medical (ISM) band from 2.4 to 2.485 gigahertz (GHz). Bluetooth® is a registered trademark owned by the Bluetooth® SIG. The communications interfaces of the network adapter 186 can also or alternatively comprise a telecommunications interface, a wireless local area network interface, one or more network interface ports, a radio-frequency (RF) coil and RF transceiver, etc.

The one or more input devices 187 are devices over which the intra-operative insertion monitoring system 110 receives input from a user. The one or more input devices 187 can include physically-actuatable user-interface elements (e.g., buttons, switches, or dials), a keypad, keyboard, mouse, touchscreen, and voice input devices, among other input devices that can accept user input. The one or more output devices 188 are devices by which the intra-operative insertion monitoring system 110 is able to provide output to a user. The one or more output devices 188 can include a display screen 190, such as a liquid crystal display (LCD), and one or more speakers 191, among other output devices for presentation of visual and/or audible information to a user (e.g., a surgeon, a clinician, an audiologist, the recipient, etc.). In certain embodiments, one or more output devices 188 (e.g., the display screen 190) and one or more input devices 187 are integrated to form a touch-screen display.

In certain embodiments, the intra-operative logic 185 stored in memory 184 comprises an insertion monitoring module 192, among other functional modules, and the processing unit 183 comprises one or more processors, microprocessors, or microcontrollers that execute instructions for the insertion monitoring module 192 stored in memory 184. That is, in one form, the insertion monitoring module 192 is implemented as software, sometimes referred to herein as insertion monitoring software, at intra-operative system 110. Therefore, when the insertion monitoring software is executed by the processing unit 183, the intra-operative system 110 is operable to perform the operations described herein. In accordance with certain embodiments presented herein, the intra-operative insertion monitoring system 110 is configured to record ECochG signals from one or more recording sites as the stimulating assembly 116 is inserted into the recipient's cochlea. More specifically, the intra-operative system 110 is configured to use electrodes 144 of the electrode array 146 to capture ECochG signals from the cochlea.

It is to be appreciated that the arrangement for the intra-operative system 110 shown in FIG. 1E is merely illustrative and that aspects of the techniques presented herein can be implemented at a number of different types of systems/devices including any combination of hardware, software, and/or firmware configured to perform the functions described herein.

As noted, elongate stimulating assembly 116 is configured to be at least partially implanted in the recipient's cochlea. During implantation of the elongate stimulating assembly 116, it is important that the insertion is atraumatic to cochlear structures and residual hearing (i.e., does not damage the delicate structures of the inner ear), and positioned appropriately (e.g., proper depth to provide adequate electrical hearing given the type of hearing loss and/or does not interfere with the biophysical mechanisms associated with acoustic hearing) for clinical benefit.

Presented herein are techniques that use real-time objective metrics in the form of measured inner ear potentials, such as acoustically-evoked responses, and/or subjective feedback to achieve these and other benefits. In particular, in certain embodiments, the intra-operative system 110 is configured to receive measured inner ear potentials from the cochlear implant system 102 during insertion of the stimulating assembly 116 into the recipient's cochlea. The measured inner ear potentials are obtained by delivering known electrical and/or acoustic stimulation to the recipient's auditory system, and recording the resulting potentials via one or more stimulating contacts 144 and one or more amplifiers 143 (FIG. 1D) located in the implantable component 112 (i.e., integrated amplifier of the cochlear implant captures one or more windows of the evoked activity). The measured inner ear potentials, which are generally represented in FIG. 1D by arrow 145, are transmitted back to the external component 104 (e.g., sound processing unit 106, surgical processor, etc.) for relay to the intra-operative system 110 or are transmitted directly to the intra-operative system 110. Using the measured inner ear potentials, the intra-operative system 110 is configured to determine whether insertion of stimulating assembly has encountered an insertion stop condition, such as a target stop condition or an error stop condition, or an insertion warning condition. Detection of one of a stop or warning condition causes the intra-operative system 110 to initiate a feedback mechanism to cause at least a temporary cessation of the insertion process.

As used herein, “inner ear potentials” refer to any voltage potential associated with either the electrical properties of the inner ear or its physiological function and/or potentials obtained via measurements at the inner ear. Potentials of a physiological nature (i.e., potentials relating to the physiological function of the inner ear), include acoustically-evoked potentials/responses (e.g., electrocochleography (ECochG) responses) and electrically-evoked potentials/responses (e.g., electrically evoked compound action potential (ECAP) responses. Other potentials of a physiological nature are referred to herein as higher evoked potentials, which are potentials related to the brainstem and auditory cortex, inclusive of the electrical auditory brainstem responses (EABR), the middle latency response, and cortical responses. Potentials of a physiological nature are sometimes referred to herein as “physiological potentials.” Potentials of electrical nature (i.e., potentials relating to the electrical properties of the inner ear itself or intra-cochlear contacts) include voltage tomography responses, measured impedances (bulk and interface), and/or other forms of electrode (stimulating contact) voltage measurements. Potentials of electrical nature are sometimes referred to herein as “physiological electrical potentials.”

As used herein, an ECochG measurement refers to the capture of a set of potentials generated in a recipient's cochlea in response to the delivery of acoustic stimulation to the cochlea. A captured set of potentials (i.e., an ECochG response) can include a plurality of different stimulus related potentials, such as the cochlear microphonic (CM), the cochlear summating potential (SP), the auditory nerve neurophonic (ANN), and the auditory nerve or Compound Action Potential (CAP), which are measured independently or in various combinations. The potentials captured as part of an ECochG response are a form of acoustically-evoked cochlear responses.

The cochlear microphonic is a fluctuating voltage that mirrors the waveform of the acoustic stimulus at low, moderate, and high levels of acoustic stimulation. The cochlear microphonic is generated by the outer hair cells of the organ of Corti and is dependent on the proximity of the recording electrode(s) to the stimulated hair cells and the basilar membrane. In general, the cochlear microphonic is proportional to the displacement of the basilar membrane by the travelling wave phenomena.

The summating potential is the direct current (DC) response of the outer hair cells of the organ of Corti as they move in conjunction with the basilar membrane (i.e., reflects the time-displacement pattern of the cochlear partition in response to the stimulus envelope). The summating potential is the stimulus-related potential of the cochlea and can be seen as a DC (unidirectional) shift in the cochlear microphonic baseline. The direction of this shift (i.e., positive or negative) is dependent on a complex interaction between stimulus parameters and the location of the recording electrode(s).

The auditory nerve neurophonic is a signal recorded from the auditory nerve, while the auditory nerve Action Potential represents the summed response of the synchronous firing of the nerve fibers in response to the acoustic stimuli, and it appears as a fluctuating voltage. The auditory nerve Action Potential is characterized by a series of brief, predominantly negative peaks, including a first negative peak (N1) and second negative peak (N2). The auditory nerve Action Potential also includes a magnitude and a latency. The magnitude of the auditory nerve Action Potential reflects the number of fibers that are firing, while the latency of the auditory nerve Action Potential is measured as the time between the onset and the first negative peak (N1).

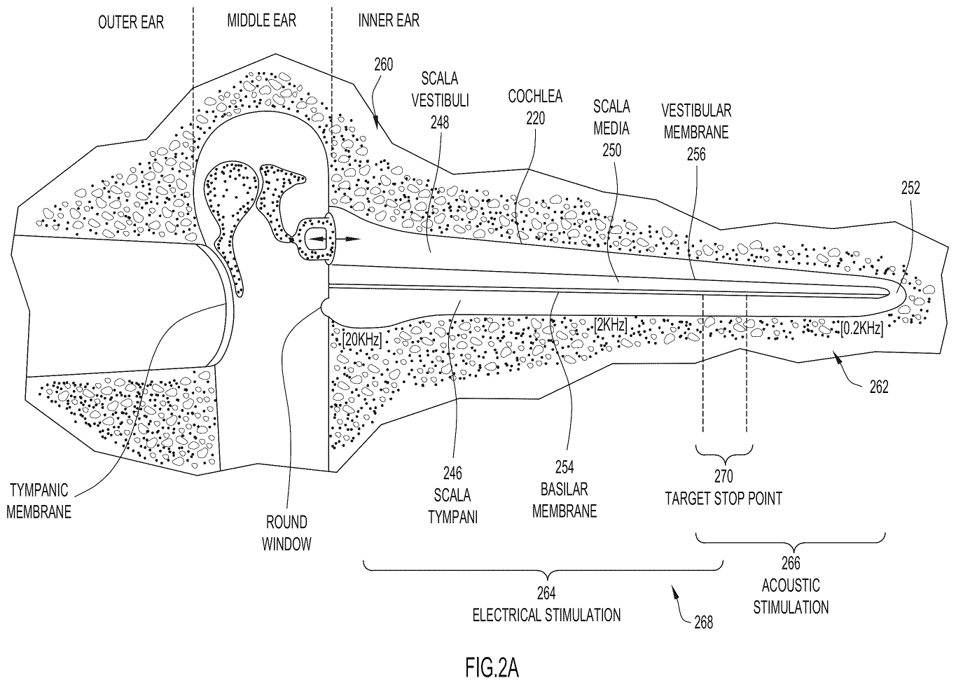

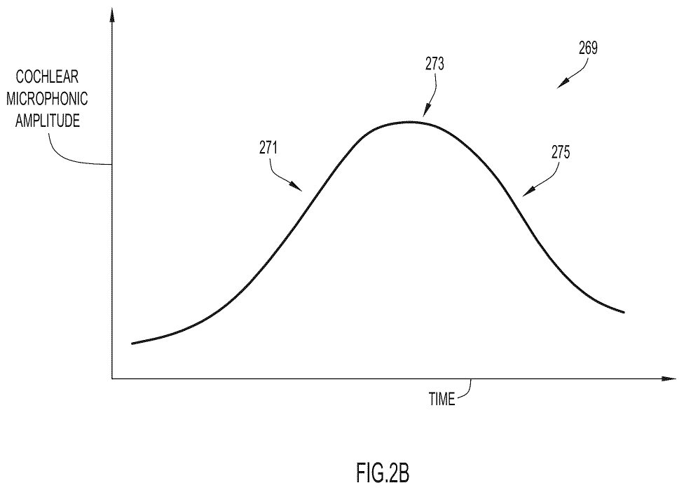

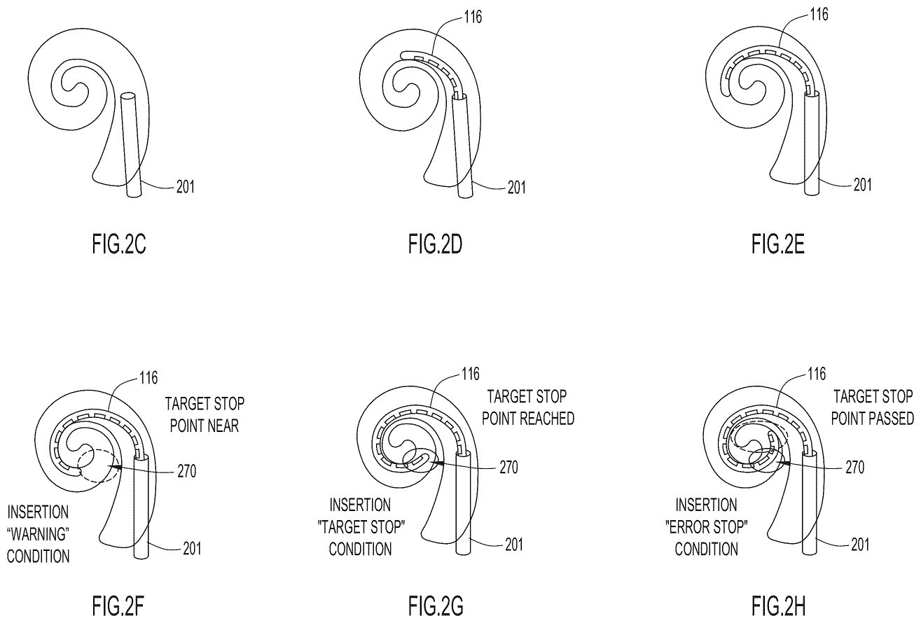

As noted above, the techniques presented herein are configured to monitor the insertion of a stimulating assembly for detection of a warning condition or a stop condition (e.g., a target stop condition and/or an error stop condition). FIG. 2A is a schematic diagram schematically illustrating the concept of a pre-operatively defined insertion stop frequency (target stop point) that corresponds to a target stop condition in accordance with embodiments presented herein.

More specifically, a recipient's cochlea is a conical spiral structure comprising three parallel fluid-filled canals or ducts, collectively and generally referred to herein as canals. For ease of illustration, FIG. 2A illustrates a cochlea 220 in an “unrolled” arrangement. The cochlea canals comprise the tympanic canal 246, also referred to as the scala tympani, the vestibular canal 248, also referred to as the scala vestibuli, and the median canal 250, also referred to as the scala media. Cochlea 220 spirals about a recipient's modiolus (not shown) several times and terminates at cochlea apex 252.

Separating the cochlea canals are various membranes and other tissue. In particular, toward a lateral side of the scala tympani 246, a basilar membrane 254 separates the scala tympani 246 from the scala media 250. Similarly, toward lateral side of the scala vestibuli 248, a vestibular membrane 256, also referred to as the Reissner's membrane, separates the scala vestibuli 248 from the scala media 250.

The scala tympani 246 and the scala vestibuli 248 are filled with a fluid, referred to herein as perilymph, which has different properties than that of the fluid which fills the scala media 248, referred to as endolymph, and which surrounds the organ of Corti (not shown). Sound entering the auricle of a recipient's ear causes pressure changes in cochlea 220 to travel through the fluid-filled tympanic and vestibular canals 246, 248. The organ of Corti, which is situated on basilar membrane 254 in scala typmani 246, contains rows of 16,000-20,000 hair cells (not shown) which protrude from its surface. Above them is the tectoral membrane which moves in response to pressure variations in the fluid-filled tympanic and vestibular canals 246, 248. Small relative movements of the layers of the tectoral membrane are sufficient to cause the hair cells in the endolymph to move thereby causing the creation of a voltage pulse or action potential which travels along the associated nerve fiber to the auditory areas of the brain for processing.

The place along basilar membrane 254 where maximum excitation of the hair cells occurs determines the perception of pitch and loudness according to the place theory. Due to this anatomical arrangement, cochlea 220 has characteristically been referred to as being “tonotopically mapped.” That is, regions of cochlea 220 toward basal region 260 are responsive to higher frequency signals, while regions of cochlea 220 toward apical region 262 are responsive to lower frequency signals. For example, the proximal end of the basal region 260 is generally responsive to 20 kilohertz (kHz) sounds, while the distal end of the apical region is responsive to sounds at around 200 hertz (Hz).

In cochlear implant recipients, residual hearing most often is present within the lower frequency ranges (i.e., the more apical regions of the cochlea) and little or no residual hearing is present in the higher frequency ranges (i.e., the more basal regions of the cochlea). This property of residual hearing is exploited in electro-acoustic hearing prostheses where the stimulating assembly is inserted into at least the basal region of the cochlea and is used to deliver electrical stimulation signals to evoke perception of higher frequency sounds. In some embodiments, insertion of the stimulating assembly is terminated before reaching the functioning regions of the cochlea where there is residual hearing so that remaining hair cells are able to naturally perceive lower frequency sounds that cause movement of the perilymph. In other embodiments, insertion of the stimulating assembly is not terminated before reaching the functioning regions of the cochlea where there is residual hearing, but the cochlear implant is configured to only deliver electrical stimulation to the more basal regions to of the cochlea and allow the remaining hair cells in the more apical regions to naturally perceive lower frequency sounds caused by movement of the perilymph. This concept is illustrated in FIG. 2A where reference 264 illustrates the region of the cochlea 220 to which electrical stimulation is delivered to evoke hearing perception, while reference 266 illustrates the region of the cochlea 220 that utilizes acoustic stimulation to evoke a hearing perception.

The tonotopic region of the cochlea 220 where the sound or stimulation output transitions from the acoustic stimulation to the electric stimulation is called the cross-over frequency region, and is illustrated in FIG. 2A by reference 268. Recipients of electro-acoustic hearing prosthesis can have different residual hearing characteristics and, accordingly, different cross-over frequency regions (i.e., transitions occur at different tonotopic regions of the cochlea). Additionally, in some instances insertion of the distal end of a stimulating assembly into and/or past the cross-over frequency region can interfere with, or damage, the recipient's residual hearing. Therefore, as noted above, an objective of the techniques presented herein is to provide a surgeon with objective measurements that enable insertion of a stimulating assembly to be halted at a depth, referred to elsewhere herein as the target stop point, that does not interfere with or damage the recipient's residual hearing and/or aligns the stimulating assembly with the natural tonotopy of the cochlea. The target stop point, which is defined as a specific frequency or a specific frequency range (pre-operatively defined insertion stop frequency), is represented in FIG. 2A by reference 270. Also as noted elsewhere herein, a target stop condition occurs when the distal end or other portion of a stimulating assembly is inserted to a location (depth) within the cochlea 220 that corresponds to (i.e., at or near) the pre-operatively defined insertion stop frequency (i.e., the specific frequency or a specific frequency range of the target stop point 270).

As noted above, the intra-operative system 110 can utilize any of a number of different types of inner ear potential measurements to determine when the stimulating assembly 116 has encountered an insertion stop condition or an insertion warning condition. FIG. 2B is a graph 269 illustrating how inner ear responses in the form of cochlear microphonic amplitudes can be used to determine when a measurement contact approaches, reaches, and passes a tonotopic region of the cochlea associated with a target stop point. The graph 269 of FIG. 2B has a vertical axis that represents the amplitude of a measured cochlear microphonic amplitudes and a horizontal axis that represents time.

In the example of FIG. 2B, the cochlea 220 of the recipient is stimulated with an acoustic input having at least one selected frequency that is associated with (i.e., corresponds to) a specific tonotopic region of the cochlea 220. As the stimulating assembly 116 is inserted into the cochlea 220, at least one contact of the stimulating assembly 116 (i.e., the measurement contact) is used to obtain ECochG measurements, which include the cochlear microphonic amplitude. As shown by reference 271 in FIG. 2B, the amplitude of the cochlear microphonic gradually increases as the measurement contact approaches the tonotopic region of the cochlea associated with the frequency of the acoustic input. As shown by reference 273, the amplitude of the cochlear microphonic peaks when the measurement contact is located at the tonotopic region of the cochlea associated with the frequency of the acoustic input. Finally, as shown by reference 275, the amplitude of the cochlear microphonic gradually decreases as the measurement contact moves away from the tonotopic region of the cochlea associated with the frequency of the acoustic input.

As noted above, an objective of the insertion process is to stop insertion of the stimulating assembly 116 when a portion of the stimulating assembly (e.g., a distal end of the stimulating assembly 116 and/or one or more stimulating contacts 144) reaches, but does not pass, a tonotopic region corresponding to the pre-operatively defined insertion stop frequency (i.e., the target stop point 270). Therefore, FIG. 2B is merely illustrative and does not represent cochlear microphonic amplitudes that would be measured in all embodiments. Instead, when the cochlear microphonic amplitude is measured at the pre-operatively defined insertion stop frequency, the insertion could stop at point 271, or ideally, at point 273 of FIG. 2B (i.e., before the measurement contact passes the target stop point 270).

In addition to a target stop condition, embodiments presented herein are also configured to monitor insertion of a stimulating assembly for warning conditions or an error stop condition. An error stop condition occurs when a stimulating assembly physically contacts or otherwise interferes with the organ of corti (including the basilar membrane 254) at any point along the stimulating assembly. Contact or interference can be due to, for example, over insertion, cochlea morphology, improper surgical trajectory, etc. Further details for detection of target stop conditions, an error stop conditions, and warning conditions are provided below.

As noted, the techniques presented herein can be used to, for example, monitor pre-curved perimodiolar electrode array insertion, and detect, in real-time (e.g., during the course of an electrode array insertion) when the electrode array approaches, reaches, and passes a target stop point (corresponding to a target frequency) within a cochlea. For example, with a perimodiolar electrode array, the electrodes should stay close to the modiolus during an optimal insertion (e.g., the electrode array moves smoothly around the modiolus, with the apical section of the electrode array maintaining close contact for the duration of the insertion). An example of this dynamic is depicted in FIGS. 2C-2H which show different stages in which an electrode array (e.g., stimulating assembly 116) is inserted in a plastic model of a cochlea using a sheath 201. FIGS. 2C-2H illustrate an electrode array trajectory in a relatively good insertion dynamic. In particular, FIGS. 2D and 2E are examples of a partial insertion with no warning condition, no target stop condition, and no error stop condition. FIG. 2F is an example of under-insertion but approaching the target stop point 270 (e.g., insertion warning condition). In the example of FIG. 2F, an alert can be generated and action can be taken based thereon (e.g., slow rate of insertion). FIG. 2G is an example of full insertion to the target stop point 270 (e.g., target stop condition). In the example of FIG. 2G, an alert can be generated and action can be taken based thereon (e.g., stop insertion). FIG. 2H is an example of over-insertion past the target stop point 270 (e.g., insertion error stop condition). In the example of FIG. 2H (over-insertion, error stop condition), an alert can be generated and corrective action can be taken based thereon (e.g., stop insertion, and at least partial retraction). After full deployment of the electrode array 116 within the cochlea, the sheath 201 is removed.

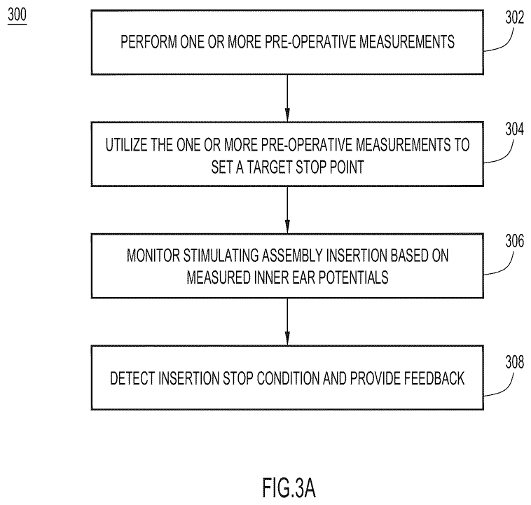

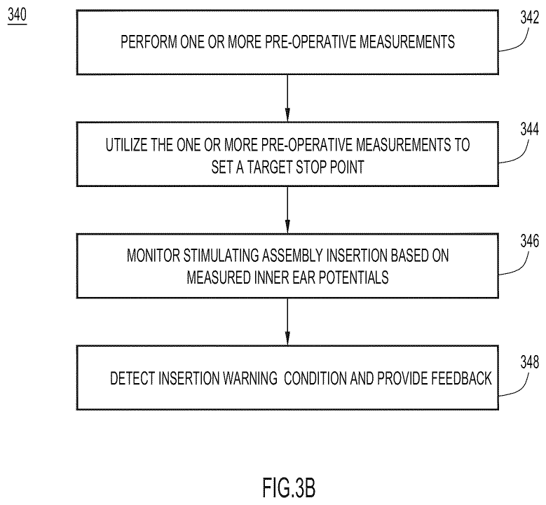

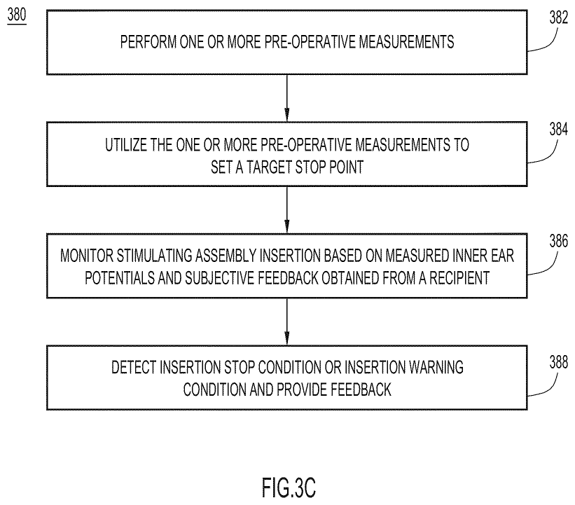

FIG. 3A is a flowchart illustrating operations associated with detection of one or more insertion stop conditions in accordance with embodiments presented herein. For ease of illustration, the method of FIG. 3A is described with reference to the intra-operative insertion monitoring system 110 and the cochlear implant system 102 of FIGS. 1A-1E.

The method 300 of FIG. 3A begins at operation 302 where one or more pre-operative tests/measurements are performed on the recipient to assess the function of cochlea 220 (i.e., the cochlea in which the stimulating assembly 116 is to be implanted). The one or more pre-operative tests can include an audiogram measurement of the recipient's cochlea 220 in order to record the recipient's residual hearing (i.e., to determine the frequency and/frequency range where the recipient's residual hearing begins). An audiogram measurement refers to a behavioral hearing test, sometimes referred to as audiometry, which generates an audiogram. The behavioral test involves the delivery of different tones, presented at a specific frequency (pitch) and intensity (loudness), to the recipient's cochlea and the recording of the recipient's subjective responses. The resulting audiogram is a graph that illustrates the audible threshold for standardized frequencies as measured by an audiometer. In general, audiograms are set out with frequency in Hertz (Hz) on the horizontal (X) axis, most commonly on a logarithmic scale, and a linear decibels Hearing Level (dBHL) scale on the vertical (Y) axis. In certain arrangements, the recipient's threshold of hearing is plotted relative to a standardized curve that represents ‘normal’ hearing, in dBHL. The audiogram is used to determine the frequency and threshold of hearing for the recipient's cochlea.

In addition to an audiogram measurement, the pre-operative tests can also include one or more imaging tests, such as a high-resolution computed tomography (CT) scan, X-ray, Magnetic resonance imaging (MRI), etc. of the recipient's cochlea. In certain embodiments, the high-resolution CT scan, and possibly the MRI, is employed clinically to determine if there are anatomical abnormities or bone growth (meningitis) prior to the surgery. The MRI can also be used to determine the viability of the auditory nerve. Moreover, the size of the cochlea can be assessed (estimated) via high-resolution CT scans that measure the anatomical landmarks, which can be used to assist with the prediction of insertion depth angles.

The one or more pre-operative tests can also include an initial inner potential measurement, such as an ECochG measurement, that is performed from outside of the cochlea (e.g., the round window). The inner potential measurements can be taken pre-operatively, using a measurement electrode that is inserted through the tympanic membrane, or intra-operatively before beginning insertion of the stimulating assembly 116 (i.e., before drilling the cochleostomy or making the incision in the round window). In the case of an ECochG measurement, ECochG responses are evoked using an acoustic input at a number of different frequencies and a fixed presentation level (e.g., supra-threshold). As such, a pre-operative ECochG measurement provides a baseline recording of the ECochG responses at each of a number of different frequencies along the length of the cochlea 220, along with the relative magnitude information between each frequency to indicate a region where maximum ECochG amplitude can be expected for each frequency.

At operation 304, the results of the pre-operative measurements (e.g., audiogram, CT scan, initial inner potential measurement, etc.) are used to set a target stop point for the stimulating assembly 116. As noted above, a target stop point is a cochlea frequency or frequency range, sometimes referred to herein as a pre-operatively defined insertion stop frequency, to which a distal end of the stimulating assembly 116 is expected to be inserted so as, for example, to be located at or near the tonotopic region where the recipient's residual hearing begins. In certain embodiments, the target stop point is set at a conservative frequency or frequency range that would minimize the probability of causing either unrecoverable or permanent damage to cochlea structures and/or the recipient's residual hearing. As such, the target stop depth is a type of predictive estimate or target that, as described below, is monitored and possibly refined during the insertion process.

In certain embodiments, normative statistics generated based on prior implantations for similar recipients can be used to further refine the target stop point (i.e., revise the target stop depth based on information determined from other recipients having similar characteristics/attributes to the subject recipient). For example, the refinement based on normative statistics can be made by taking into account the recipient's age (i.e., refine based on implantation results from similarly aged recipients), the one or more imaging tests (e.g., based on implantation results for recipient's having similar X-rays, CT scans, etc.), hearing loss, etiology, or other shared characteristics.

In addition to the target stop point, the results of the pre-operative measurements can also be used to set a minimum insertion depth. The minimum insertion depth defines the depth to which the distal end of the stimulating assembly 116 is estimated to be implanted in the cochlea in order to provide acceptable electrical-only hearing performance (e.g., based on existing clinical evidence). This minimum insertion depth would take into account anatomical differences (e.g., smaller cochlea sizes, malformations, etc.) identified by the pre-operative measurements. In general, the minimum insertion depth could be specified as a frequency, frequency range, or an angle. Defining the minimum insertion depth as an angle takes into account the variations in the cochlea anatomical size and the electrode type (modiolar or lateral wall), etc.

In general, it has been determined that for electrical-only hearing (i.e., only electrical stimulation) with a full-length array, there is a minimum insertion depth where maximum clinical benefit can be obtained. Any insertion depth that is under this is distance, again for electrical-only hearing, may have a poor clinical outcome. It is also expected that, for some recipients, residual acoustic hearing will eventually reduce, leaving only the electrical hearing abilities. For recipients likely willing to undergo revision surgery (e.g., children), the minimum insertion depth can be an insertion depth where it is ensured that the acoustic hearing is fully unperturbed by the introduction of the stimulating assembly. For recipients unlikely to undergo an additional surgery (e.g., older recipients), the minimum insertion depth can be a depth where it is determined that, when the residual acoustic hearing deteriorates, the implanted stimulating assembly will still provide acceptable electrical-only performance.

After setting the target stop point and the minimum insertion depth, the surgeon begins implantation of the stimulating assembly (e.g., opens the cochleostomy or incises the round window and inserts the distal end into the cochlea). At operation 306, the insertion of the stimulating assembly 116 is monitored using objective inner ear potentials measured, in real-time, via one or more stimulating contacts 144. For example, in certain embodiments one or more acoustic tones (e.g., pure tone(s)) at a selected frequency or frequencies are delivered to the recipient's outer ear using, for example, the receiver 132. The acoustic signals delivered by the receiver 132 cause displacement waveforms that travel along the basilar membrane. These waves grow in amplitude and reach a maximum at the characteristics frequency (CF) as a function of frequency along the cochlea. These vibrations along the cochlea give rise to an inner ear potential. Therefore, in response to the delivered acoustic signals, one or more of the stimulating contacts 144 and the integrated amplifier(s) 143 of the cochlear implant 112 capture one or more windows of the evoked activity (i.e., perform ECochG measurements) to generate inner ear response measurements (e.g., ECochG response measurements) that are provided to the intra-operative system 110. In other words, the intra-operative system 110 monitors the inner ear response at one or more of the stimulating contacts 144.

In certain examples, the acoustic signals delivered by the receiver 132 of the hearing aid 131 are selected based on the results of the one or more pre-operative tests. For example, the acoustic signals can have a frequency that is the same as, or close to, the frequency of the target stop point (e.g., at a frequency where the augmented hearing (electrical and acoustic) offers the maximal clinical benefit, around a predetermined cutoff frequency where acoustic hearing starts, etc.,) determined from the pre-operative audiogram.

At operation 308, an insertion stop condition is detected and, in response to the detection, a feedback mechanism is initiated/triggered. As noted above, an insertion stop condition can include a target stop condition, meaning that the intra-operative system 110 has determined that a selection portion of the stimulating assembly 116 (e.g., distal end, one or more stimulating contacts 144, etc.) has reached the tonotopic region corresponding to the frequency defining the target stop point. If the target stop condition is detected, insertion of the stimulating assembly 116 should be terminated to prevent damage to the cochlea and/or the recipient's residual hearing.

Also as noted above, an insertion stop condition can also be an error stop condition, meaning that the stimulating assembly 116 has interfered with (e.g., come into physical contact with) an intra-cochlea structure, such as the basilar membrane. For example, it is possible that the stimulating assembly can move outwards towards the scala wall, at points other than the apical region of the stimulating assemble (e.g., a modiolar hugging array, which is flexible, can reach a point in the insertion when it meets physical resistance and bows outwards to the scala wall, riding up the wall to eventually make physical contact with the basilar membrane). If an error stop condition is detected, corrective action should be initiated.

In accordance with the embodiments presented herein, the insertion stop condition(s) or the insertion warning condition(s) can be detected in a number of different manners. More specifically, referring first to the detection of a target stop condition, one or more objective inner ear potential measurements, such as ECochG measurements, are continually performed in real-time while the stimulating assembly 116 is inserted into the cochlea 220 (i.e., as the stimulating assembly 116 is moved in an apical direction). The intra-operative system 110 analyzes the measured real-time inner ear potentials relative to one another to determine if a change in the measured response occurs. The change can be, for example, an expected change in the magnitude/amplitude, phase, shape of the response/waveform (morphology), frequency, or other aspects of the responses. For example, in one embodiment, an expected change indicative of a target stop condition comprises the detection of a peak or near peak in acoustically-evoked inner ear potentials (e.g., CM components of ECochG responses).

Again, in accordance with embodiments presented herein, the real-time inner ear potential measurements can be made in a number of different manners at one or more locations (e.g., simultaneously, sequentially, etc.) within the cochlea. In certain embodiments, inner ear potential measurements can be used to monitor or track the progression of the stimulating assembly 116 within the cochlea 220 using one or more complex acoustic inputs (sound signals) comprising multiple frequencies. In other words, inner ear potential measurements can be performed based on several different acoustic frequencies and/or at different contacts of the stimulating assembly (i.e., multi-frequency acoustic inputs and/or multi-electrode/contact recording). The complex acoustic inputs can comprise, for example, a frequency sweep signal, a series of sound chirps, etc.

More specifically, the intra-operative system 110 can be configured to perform inner ear potential measurements at any of a number of contacts (in response to the same or different acoustic input), and then perform a comparison relative to another and/or against previous time points (e.g., pattern matching, correlation, etc.). Signal features that can be compared at, for example, different time points (cross-time point comparison) include phase, amplitude, morphology, etc. Based on the comparisons, the intra-operative system 110 could determine, for example, current insertion depth, location relative to the predicted depth, basilar membrane contact, stimulating assembly deformation (e.g., bowing), tonotopic mapping in the cochlea, changes to the acoustic resonant properties of the cochlea, etc.

In summary, the inner ear potential measurements in accordance with embodiments presented herein can make use of multi-electrode recording, complex acoustic inputs, cross-time comparisons, and/or cross-electrode comparisons. These variations could, for example, enable the system to gauge the current tonotopic position of the measurement contact(s) (e.g., apical electrode) and/or to set a baseline that makes early-stage detection of a “shift” in the cochlea microphonic (such as a change in resonant frequency) easier to detect.

In certain embodiments, one or more complex acoustic inputs (e.g., a frequency sweep signal or a series of sound chirps) are used to correlate the position of one or more contacts with a frequency response as the array advances within the cochlea. That is, the one or more complex acoustic inputs enable the intra-operative system 110 to detect the current tonotopic position of one or more contacts and, accordingly, the position of the stimulating assembly 116. In one such example, one or more complex acoustic inputs are delivered to the cochlea 220 to evoke responses along the cochlea at tonotopic locations preceding the target frequency (i.e., frequencies that are higher than the target stop point). Using these responses, the intra-operative system 110 can then determine when the measurement contacts (e.g., the apical contact) approaches, reaches, and passes the tonotopic region of the cochlea associated with each of the frequencies. As noted above, FIG. 2B illustrates an example of a cochlear microphonic amplitude measured as a measurement contact approaches, reaches, and passes the tonotopic region of the cochlea 220 that corresponds to the frequency of an acoustic input.

In one specific example, one or more complex acoustic inputs having specific frequency components of at least 2 kHz, 1.5 kHz, 1 kHz, and 500 Hz are presented to the cochlea 220. In this example, the target stop point is 500 Hz. As the stimulating assembly 116 is inserted into the cochlea 220, ECochG measurements are performed at the most apical contact to obtain cochlear microphonic amplitudes for each of the tonotopic regions corresponding to the frequencies in the one or more complex acoustic inputs (e.g., at least 2 kHz, 1.5 kHz, 1 kHz, and 500 Hz). During insertion towards the target 500 Hz, the apical contact will approach, reach, and pass the tonotopic regions associated with 2 kHz, 1.5 kHz, 1 kHz, thereby rendering cochlear microphonic amplitudes similar to that shown in FIG. 2B (i.e., a rise, peak, and fall) at each of the corresponding tonotopic regions. Using the cochlear microphonic amplitudes measured at one or more of the tonotopic regions preceding the target stop point, the intra-operative system 110 could, for example, refine the target stop point, determine a dynamic trigger indicating that the target stop point has been reached, track the location of the apical contact, etc. In one specific example, the frequency band of the acoustic input can be reduced to frequencies adjacent the target frequency as the insertion depth approaches the target depth to give the system greater resolution.

For example, the cochlear microphonic amplitude peaks at two sequential tonotopic regions of the cochlea 220 (e.g., the 2 kHz region and the 1.5 kHz region) could be detected and analyzed relative to one another for translation into a distance measurement. That is, the intra-operative system 110 could, using a scaling of the cochlea, determine that the movement between two of the higher frequencies equates to a certain distance. Through multiple data points and cochlear scale, the intra-operative system 110 can determine how far past the 1 kHz region the stimulating assembly 116 should be inserted so as to reach the 500 Hz region. Accordingly, using these measurements, the intra-operative system 110 could then trigger a notification when the apical contact reaches or gets close to the 500 Hz region. In summary, the intra-operative system 110 could use the responses measured at earlier (higher) frequencies to determine and measure cochlea distances and, accordingly, improve the insertion process.

In another example, cochlear microphonic amplitude peaks detected at earlier sequential tonotopic regions of the cochlea 220 (e.g., the 2 kHz, 1.5 kHz, and 1 kHz regions) could be used to determine when the cochlear microphonic amplitude is approaching a peak, or has reached the peak, at the 500 Hz region. More specifically, the earlier cochlear microphonic amplitude peaks could be correlated with one another (e.g., pattern matching) to generate one or more recipient-specific “peak characteristics.” As the apical contact approaches the 500 Hz, the intra-operative system 110 could analyze the measured cochlear microphonic for these recipient-specific peak characteristics and, accordingly, trigger a notification when the apical contact reaches or gets close to the 500 Hz region.

As noted above, embodiments presented herein can also make use of multi-electrode/contact recording. In one form, the multi-electrode recording enables the intra-operative system 110 to monitor patterns. For example, the intra-operative system 110 could record inner ear potential measurements for each frequency component at successive measurement contacts. If the acoustic input remains the same, the response recorded at each successive contact could be correlated with one other. In one example, deviations in the response profile across the cochlea could be indicate initial changes to the basilar membrane impedance.

The multi-electrode recording could also be used to gauge insertion depth from the neural response profile and, accordingly, prevent the stimulating assembly from passing the target stop point. In one example, each time a successive electrode contact passes an established neural response peak, the intra-operative system 110 can increment a cumulative insertion depth total by the distance between adjacent electrode contacts. Further to this, the insertion depth can be approximated by a pattern matching procedure whereby subsequent measurement contacts (non-apical) that pass by the earlier apical recording position are matched to provide a relative measure between measurement contacts. Given that the distance between contacts is known this, this distance can be then converted into an insertion depth measure.

In certain embodiments, multi-electrode recording can be used to capture transimpedance measurements (e.g., to generate a Transimpedance Matrix) that, when coupled with an appropriate algorithm, can be used to estimate physical depth of insertion. This information can be used as an additional reference of the angular depth of the electrode to corroborate the comparison with the acoustic frequency stimulation.

In certain embodiments, the intra-operative system 110 begins to monitor the inner ear potentials for changes when the distal end of the stimulating assembly 116 approaches the minimum insertion depth, where the forward movement of the stimulating assembly 116 is slowed. In other embodiments, the intra-operative system 110 begins to monitor the inner ear potentials for changes as soon as the one or more contacts used for measurement of the potentials enter the cochlea. Other starting points can be used in alternative embodiments.

In the event that a change in the measured inner ear potentials is detected, the intra-operative system 110 could trigger insertion of the stimulating assembly 116 to be halted for a period of time (e.g., stop automated insertion process, generate a notification to a surgeon, etc.). When the insertion is halted, the real-time inner ear potential measurements are continued and the intra-operative system 110 continues to monitor the measured potentials to determine if they return/recover to (i.e., to determine whether the identified change disappears). If no recovery is detected, then the intra-operative system 110, surgeon, etc. could initiate on or more remedial actions. For example, if it is determined that the distal end of the stimulating assembly 116 has already passed the minimum insertion depth, then the intra-operative system 110 could determine that the target stop point has been reached (i.e., the target stop condition has occurred). As a result, insertion of the stimulating assembly 116 is ceased and the stimulating assembly is secured with the recipient. In other examples, a change in the measured inner ear potentials (e.g., a sharp drop in the cochlear microphonic) that doesn't recover is likely to indicate contact with the basilar membrane (i.e., an error stop condition) that can be addressed with corrective action as described elsewhere herein.

As noted, the intra-operative system 110 is configured to monitor the measured inner ear potentials to determine if a change occurs (e.g., detects a change in the ECochG responses). In the event that no change is observed, the intra-operative system 110 compares the measured inner ear potentials at one or more acoustic frequencies against the pre-insertion inner ear potential response (e.g., the baseline). If, on comparison, the measured inner ear potentials approach the pre-insertion inner ear potential response, then insertion of the stimulating assembly 116 is halted. If the target stop point has been reached, then the stimulating assembly 116 is secured within the recipient.

In certain embodiments, when the target stop point has been reached, the measured inner ear potentials would begin to decrease slightly (i.e., the measurement contact has begun to pass the excitory location). In the event the measurement contact has not reached the target stop point, the signal is still increasing (i.e., still yet to reach the maximum value which is the target stop point).

An error stop condition occurs when the intra-operative system 110 detects a significant change (e.g., magnitude reduction) in the measured inner ear potentials during the insertion before the target stop point has been reached. In this scenario, the insertion of the stimulating assembly 116 is halted for a period of time to determine if the measured inner ear potentials recover. If no recovery is detected, then corrective action is initiated.

For example, in one specific example of a corrective action, the stimulating assembly 116 can be partially retracted and the system monitors the cochlear to determine if the measured inner ear potentials recover. If no recovery is detected, then the intra-operative system 110, surgeon, etc. could initiate one or more remedial actions. For example, the stimulating assembly 116 could be further retracted and re-inserted using a new trajectory, the surgical plan could be revised to call for a full insertion, etc.

For example, a sharp drop in the magnitude (amplitude) of the cochlea microphonic (CM) in measured ECochG responses can indicate contact with the basilar membrane. More specifically, if the stimulating assembly physically contacts the basilar membrane, this contact changes the mechanical impedance of the basilar membrane which, in turn, reduces the magnitude of the CM (e.g., a physical impingement of the basilar membrane or partition will shift the resonant peak of the cochlea microphonic). This can be reversible by partially retracting the stimulating assembly to remove the mechanical impedance. However, contact with the basilar membrane can also, in certain circumstances, indicate a dynamic stop depth where greater insertion is likely to damage or further impede movement of the basilar membrane. It is also noted that the nature of the contact with the basilar membrane can be reliant on the stimulating assembly type in use. For example, a perimodiolar electrode can bow outwards at the back wall of the cochlea when nearing over insertion. As such, the techniques presented are configured to capture the inner ear potentials at different points along the array not just the apical contact. At any point along the insertion, if the intra-operative system 110 detects a shift in the cochlea microphonic, which can indicate contact with the internal structure of the cochlea (e.g., basilar membrane), then the system triggers an alert to stop the insertion. A premature stop (i.e., prior to the target stop point) can cause the surgeon to take some corrective action, such as retract the assembly, re-insert with a different trajectory, etc.

As noted above, in addition to the stop conditions, embodiments of the present invention can also detect one or more insertion warning (i.e., slow down) conditions. An insertion warning condition occurs when the system determines that insertion of the stimulating assembly is approaching, or is likely to soon satisfy, one of the insertion stop conditions. For example, an insertion warning condition can be detected by, for example, identifying, the start of a rise in, or a steady rise in, the CM magnitude indicating an approach to the peak (for the target stop point) or the start of a drop in the CM magnitude (for the error stop point). In other examples, an insertion warning condition could be detected using the multi-frequency or multi-electrode recording techniques described elsewhere herein. Regardless of how detected, the detection of an insertion warning condition can result in the initiation of a feedback mechanism indicating that insertion of the stimulating assembly into the cochlea should be slowed (e.g., to slow automated forward movement of a stimulating assembly, an alert or notification to a surgeon to slow forward movement, etc.).

FIG. 3B is a flowchart illustrating operations associated with detection of one or more insertion warning conditions in accordance with embodiments presented herein. The method 340 of FIG. 3B begins at operation 342 where one or more pre-operative tests/measurements are performed on the recipient to assess the function of cochlea 220 (i.e., the cochlea in which the stimulating assembly 116 is to be implanted). At operation 344, the results of the pre-operative measurements (e.g., audiogram, CT scan, initial inner potential measurement, etc.) are used to set a target stop point for the stimulating assembly 116. At operation 346, the insertion of the stimulating assembly 116 is monitored using objective inner ear potentials measured, in real-time, via one or more stimulating contacts 144. The details of operations 342, 344, and 346 of the method 340 of FIG. 3B are the same or similar as described above with reference to operations 302, 304, and 306 of the method 300 of FIG. 3A. At operation 348, an insertion warning condition is detected and, in response to the detection, a feedback mechanism is initiated/triggered.

As noted above, if the insertion warning condition is detected, the feedback mechanism indicates that the automated or manual insertion of the stimulating assembly 116 should be slowed, for example. This example embodiment can give advance notice that the stimulating assembly is getting close to the target stop point. In certain examples, the monitoring can detect a slightly higher frequency than the target stop condition, which would be reached in an intermediate region before the target stop point. This use of an insertion warning condition would help to avoid over-insertion of the stimulating assembly 116 (stimulating electrodes 144), since the surgeon or surgical robot would know that they are nearing the insertion warning condition before the insertion stop condition is reached.

In another variation of the method 300 of FIG. 3A and/or the method 340 of FIG. 3B, the feedback mechanism could be further extended to refine the position of the stimulating assembly 116 via a “pull-back” (i.e., retract, reverse the direction of insertion, etc.). This variation can address a situation where a “stop” might be delayed, resulting with the stimulating assembly 116 (electrodes 144) being inserted deeper than the target stop point. That is, the pull-back would refine the position to be more precisely at the intended target stop frequency.

In many cases, hearing preservation surgery is done with an awake patient under sedation and local anesthesia. In these cases, the patient can report on whether electrical stimulation from the electrode approaches or matches the target frequency. For example, this can be done by setting the stimulus frequency for ECochG measurements to the target stop frequency, such that an acoustic stimulus followed by an electrical stimulus is delivered to the recipient in repeated bursts, and the recipient can compare the respective stimuli and advise if they are the same or different. This use of “subjective feedback” from the recipient (i.e., to supplement the “objective” measurements) would add confidence that the stimulating assembly 116 (the stimulating electrodes 144) has been inserted to the intended target frequency (target stop point). This concept can also be extended to an early warning frequency (FIG. 3B), in addition to a target stop frequency or an error stop frequency (FIG. 3A), which can give additional confidence regarding whether the intended target has been reached.

In certain cases, it may be beneficial to use different stimulus arrangements to triangulate on a result. For example, it may be beneficial to use a fixed frequency acoustic input and then rove the electrode stimulus to find which electrode corresponds to the acoustic frequency. Alternatively, it may be beneficial to have a fixed electrical stimulus and rove the frequency of the acoustic stimulus to confirm the match between electrode and acoustic stimuli. Other arrangements are also possible.

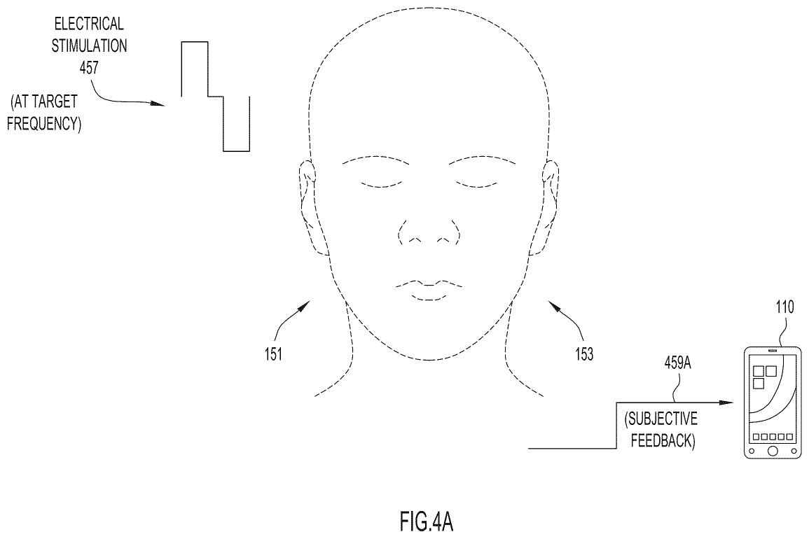

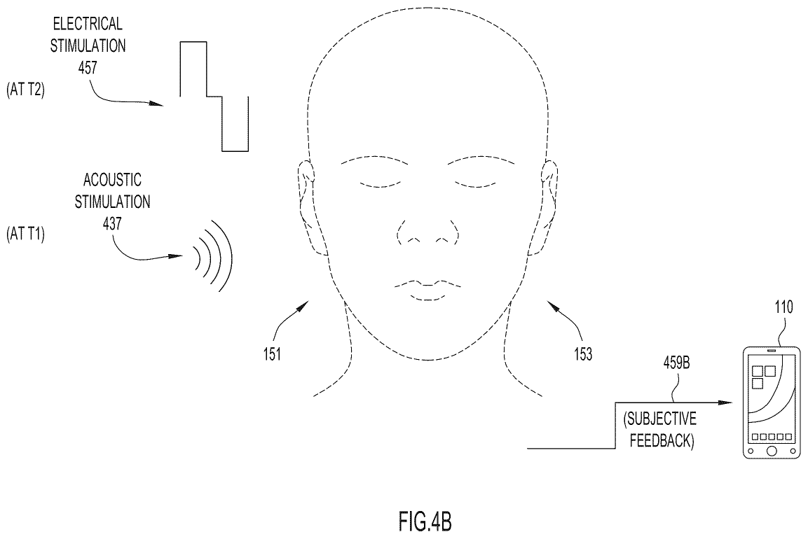

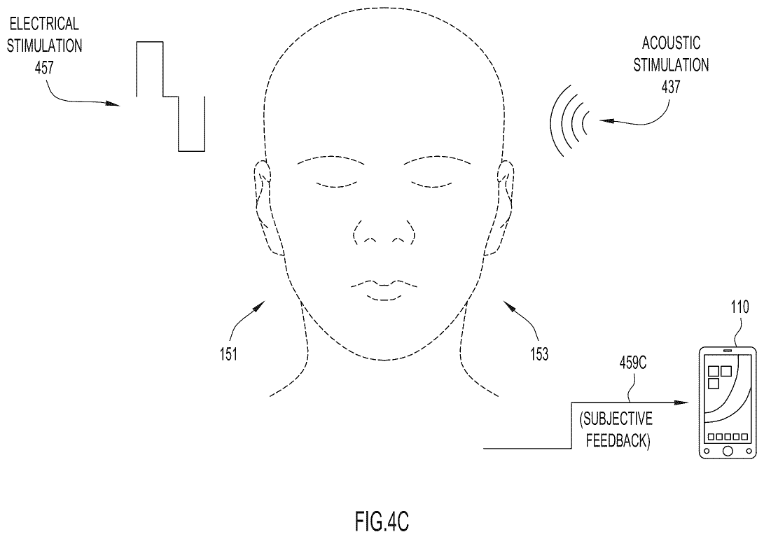

FIG. 3C is a flowchart illustrating operations associated with detection of at least one of an insertion stop condition (e.g., an insertion target stop condition or an insertion error stop condition) or an insertion warning condition using subjective feedback, in accordance with embodiments presented herein. FIG. 4A is a schematic diagram illustrating one example of the method 380 of FIG. 3C. For ease of description, FIG. 3C and FIG. 4A will be described together.

The method 380 of FIG. 3C begins at operation 382 where one or more pre-operative tests/measurements are performed on the recipient to assess the function of cochlea 220 (i.e., the cochlea in which the stimulating assembly 116 is to be implanted). At operation 384, the results of the pre-operative measurements (e.g., audiogram, CT scan, initial inner potential measurement, etc.) are used to set a target stop point for insertion of the stimulating assembly 116 into a first ear 151 (FIG. 4A) of the recipient. The details of operations 382 and 384 of the method 380 of FIG. 3C are the same or similar as described above with reference to operations 302 and 304 of the method 300 of FIG. 3A.

At operation 386, the insertion of the stimulating assembly 116 is monitored using objective inner ear potentials measured, in real-time, via one or more stimulating contacts 144, and further based on subjective feedback obtained from a recipient during insertion of the stimulating assembly 116 into the cochlea of the recipient. For ease of illustration, stimulating assembly 116 and the stimulating contacts 144 are omitted from FIG. 4A, but FIG. 4A does schematically illustrate delivery of electrical stimulation signals 457 to the cochlea of a first ear 151 (e.g., via the stimulating assembly 116 of the cochlear implant 112). As noted elsewhere herein, when these measured inner ear potentials are captured (e.g., in response to the electrical stimulation signals 457), subjective feedback 459A is also obtained from the recipient to monitor insertion, and a determination is made with respect to the insertion status based on these objective measurements and further based on the subjective feedback 459A obtained from the recipient. At operation 388, at least one of an insertion stop condition or an insertion warning condition is detected (i.e., based on the objective measurements and/or the subjective feedback 459A) and, in response to the detection, a feedback mechanism is initiated/triggered.

For example, the electrical stimulation signals 457 can be set to the target stop frequency to deliver a corresponding tone to the cochlea of the recipient, and the subjective feedback 459A indicates whether the recipient is able to detect the tone corresponding to the target stop frequency. If the subjective feedback 459A indicates that the recipient cannot hear this tone, then the stimulating assembly 116 could be under-inserted (e.g., warning condition) or possibly over-inserted (e.g., error stop condition), for example. In the case of under-insertion, the feedback mechanism can indicate that insertion should be slowed. In the case of over-insertion, the feedback mechanism can indicate that insertion should be stopped and at least partially retracted. If the subjective feedback 459A indicates that the recipient can hear this tone that corresponds to the target stop frequency, however, then the stimulating assembly 116 is confirmed to be inserted to the intended target depth in the cochlea (e.g., target stop condition). In the case of full insertion, the feedback mechanism can indicate that the insertion should be stopped. This example embodiment can utilize the subjective feedback together with the objective measurements to provide a more personalized tonotopic mapping to account for different variations in individual recipients, for example, and thereby improve accuracy of the intra-operative insertion monitoring process.

FIG. 4B is a schematic diagram of another example of the method 380 of FIG. 3C illustrating the use of subjective feedback obtained from a recipient, in combination with objective measurements as described herein, to monitor insertion of a stimulating assembly 116 into a cochlea of a recipient. In FIG. 4B, acoustic stimulation signals 437 are delivered to the cochlea of the first ear 151 (e.g., via the receiver 132 of the hearing aid 131) during a first time period (T1), and electrical stimulation signals 457 are delivered to the cochlea of the first ear 151 (e.g., via the stimulating assembly 116 of the cochlear implant 112) during a second time period (T2), or vice versa in other embodiments. In this example, the acoustic stimulation signals 437 are set to the target stop frequency to deliver a corresponding tone to the cochlea of the recipient, and subjective feedback 459B obtained from the recipient indicates whether a tone heard by the recipient as a result of delivering the electrical stimulation signals 457 during the second time period (T2) matches the tone heard by the recipient as a result of delivering the acoustic stimulation signals 437 during the first time period (T1).

In the example of FIG. 4B, if the subjective feedback 459B indicates that the recipient hears different tones (i.e., different pitches) resulting from delivery of the respective stimulation signals to the same ear during T1 and T2, respectively, then the stimulating assembly 116 could be under-inserted (e.g., warning condition) or possibly over-inserted (e.g., error stop condition), for example. In the case of under-insertion, the feedback mechanism can indicate that insertion should be slowed. In the case of over-insertion, the feedback mechanism can indicate that insertion should be stopped and at least partially retracted. If the subjective feedback 459B indicates that the recipient hears the same tone (i.e., matching pitches) resulting from delivery of the respective stimulation signals to the same ear during both T1 and T2, however, then the stimulating assembly 116 is confirmed to be inserted to the intended target depth in the cochlea (e.g., target stop condition). In the case of full insertion, the feedback mechanism can indicate that the insertion should be stopped.

FIG. 4C is a schematic diagram of yet another example of the method 380 of FIG. 3C illustrating the use of subjective feedback obtained from a recipient, in combination with objective measurements as described herein, to monitor insertion of a stimulating assembly 116 into a cochlea of a recipient. In FIG. 4C, acoustic stimulation signals 437 are delivered to the cochlea of a second ear 153 of the recipient (e.g., via the receiver 132 of the hearing aid 131), and electrical stimulation signals 457 are delivered to the cochlea of the first ear 151 of the recipient (e.g., via the stimulating assembly 116 of the cochlear implant 112). In this example, the acoustic stimulation signals 437 are set to the target stop frequency to deliver a corresponding tone to the cochlea of the recipient, and subjective feedback 459C obtained from the recipient indicates whether a tone heard by the recipient as a result of delivering the electrical stimulation signals 457 to the first ear 151 matches the tone heard by the recipient as a result of delivering the acoustic stimulation signals 437 to the second ear 153. In this example, the acoustic stimulation signals 437 and the electrical stimulation signals 457 can be delivered simultaneously, or the acoustic and electrical stimulation can alternate so as to be delivered successively (at different times).

In the example of FIG. 4C, if the subjective feedback 459C indicates that the recipient hears different tones (i.e., different pitches) resulting from delivery of the respective stimulation signals to the two different ears, respectively, then the stimulating assembly 116 could be under-inserted (e.g., warning condition) or possibly over-inserted (e.g., error stop condition), for example. In the case of under-insertion, the feedback mechanism can indicate that insertion should be slowed. In the case of over-insertion, the feedback mechanism can indicate that insertion should be stopped and at least partially retracted. If the subjective feedback 459C indicates that the recipient hears the same tone (i.e., matching pitches) resulting from delivery of the respective stimulation signals to the two different ears, however, then the stimulating assembly is confirmed to be inserted to the intended target depth in the cochlea (e.g., target stop condition). In the case of full insertion, the feedback mechanism can indicate that the insertion should be stopped.

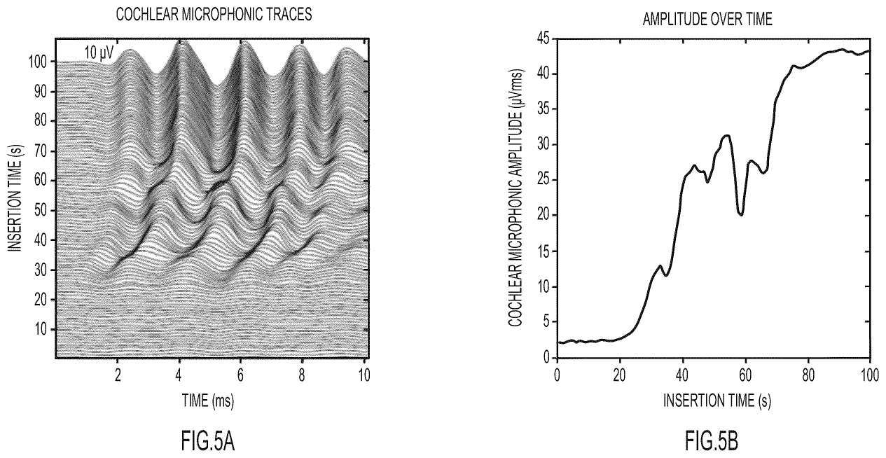

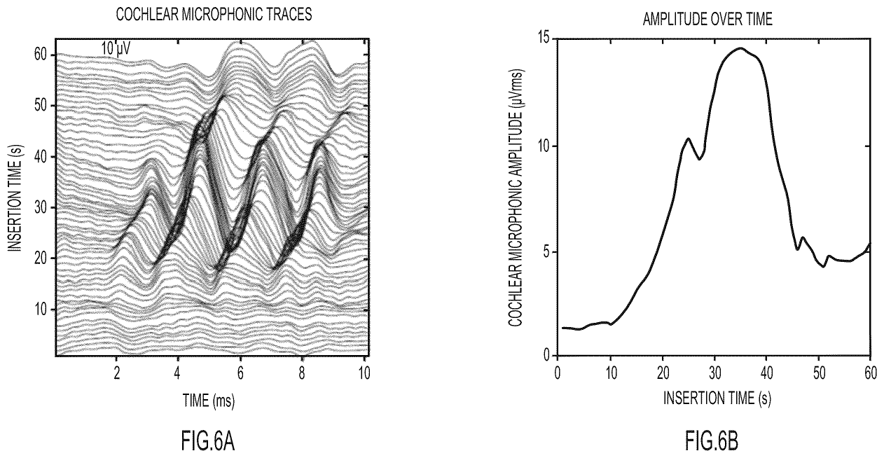

FIGS. 5A-5B and FIGS. 6A-6B illustrate the detection of an error stop condition using the magnitude of the cochlea microphonic in a measured ECochG response. More specifically, FIGS. 5A-5B and FIGS. 6A-6B illustrate cochlear microphonic traces at approximately 1 per second, when a stimulating assembly is inserted in two different recipients. FIGS. 5A and 5B illustrate an atraumatic insertion where the cochlear microphonic amplitude increases with insertion depth as the stimulating assembly approaches the target stop point. However, FIGS. 6A and 6B illustrate a more traumatic insertion where the cochlear microphonic amplitude initially increases with depth, but then significantly drops when the stimulating assembly contacts the basilar membrane.

In certain embodiments, the target stop point can be refined dynamically during the insertion when, for example, the measured inner ear potentials (measured during insertion) suggest the procedure to be an outlier to that which was originally expected. An example might be a surgery where the insertion reaches beyond the pre-operatively determined stop point and the cochlear microphonic signal is continuing to increase rapidly. Here the surgeon would continue to insert the array past this point until the insertion reaches a revised stop point determined by a slight decrease in the cochlear microphonic.

In the examples of FIGS. 3A-3C, one or more pre-operative measurements (e.g., an audiogram) are performed to determine where the recipient's residual hearing starts. This information can be used to set a target location to which a distal end of a stimulating assembly is to be inserted so that the distal end is positioned close to the boundary where the residual hearing begins. While the stimulating assembly is inserted, an acoustic input is delivered to the cochlea and the system measures ECochG responses of the cochlea cells to the acoustic input. These real-time measurements can be utilized to determine when the distal end of the stimulating assembly approaches the target location (target stop point) and/or to determine if the stimulating assembly contacts or otherwise interferes with the basilar membrane during the insertion process. Such an approach marries the functional/behavioral hearing, as indicated by the audiogram, with electrophysiological/objective measurements, as indicated by the ECochG responses, to provide a customizable insertion depth stopping point for each recipient.

In a specific example in accordance with embodiments presented herein, an audiogram is performed on a recipient and indicates that a recipient has useable residual beginning around, and below, approximately 500 Hz. As such, a stimulating assembly should be inserted in this recipient such that the distal end advances close to the 500 Hz region, but not beyond this region. In order words, 500 Hz is entered as the target stop point for insertion of the stimulating assembly.

During insertion, a 500 Hz acoustic signal is delivered to the recipient's cochlea, and the cochlear implant 112 performs ECochG measurements via one or more contacts (electrodes) within the cochlea. The intra-operative system 110 monitors the ECochG responses generated from the measurements. As the one or more contacts approach the 500 Hz region of the cochlea, an increase in the ECochG amplitude is detected. The reason for this increase is that the one or more contacts move closer to the point of excitation (i.e., the 500 Hz region). When the one or more contacts reach the 500 Hz region, a peak in the ECochG response amplitude occurs. However, if the one or more contacts move past the region 500 Hz region, the ECochG response amplitude begins to drop. In certain such embodiments, the CM amplitude is utilized, where the CM is a phase following response. However, other ECochG components or attributes, such as the phase, shape of the waveform (morphology), frequency, etc. could be used in alternative embodiments.

As noted above, the techniques presented herein can provide surgeons or other users with an understanding of surgically recorded ECochG signals and the implications for surgeons. That is, the intra-operative insertion monitoring system 110 is configured to automatically analyze the ECochG signal data and provide surgeons or other users with guidance as to when to perform a surgical intervention to maximize/balance hearing preservation and electrode insertion depth (e.g., provide surgeons with information to guide their decision process and maximize preservation of residual hearing and overall outcomes). In one example, the intra-operative system 110 is configured such that an alarm/alert is generated when the system detects a significant disruption to acoustic hearing, but no alarm/alert when the system determines a cochlear microphonic magnitude change due to local anatomy. In specific examples, the system does not sound an alarm if the stimulating assembly is retracted to a point that before had a lower cochlear microphonic magnitude. Similarly, if the surgeon withdraws the stimulating assembly to recover from a drop and goes past the location of the peak response, the system takes into consideration the new location when determining if the drop has recovered.

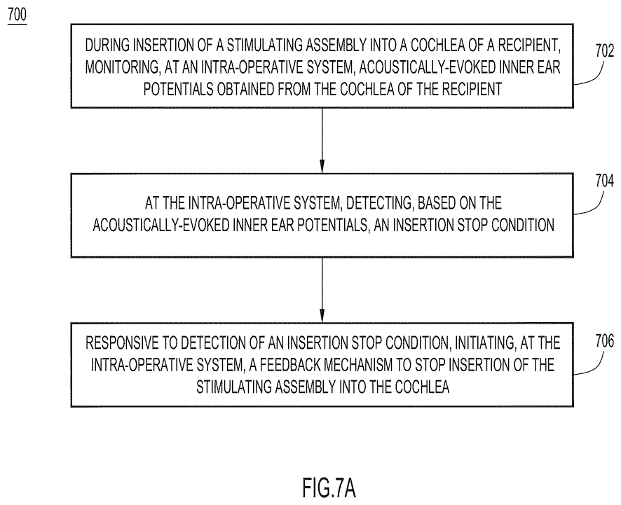

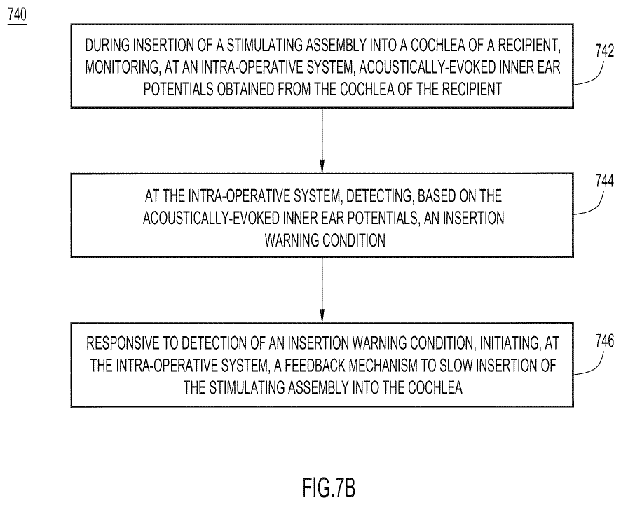

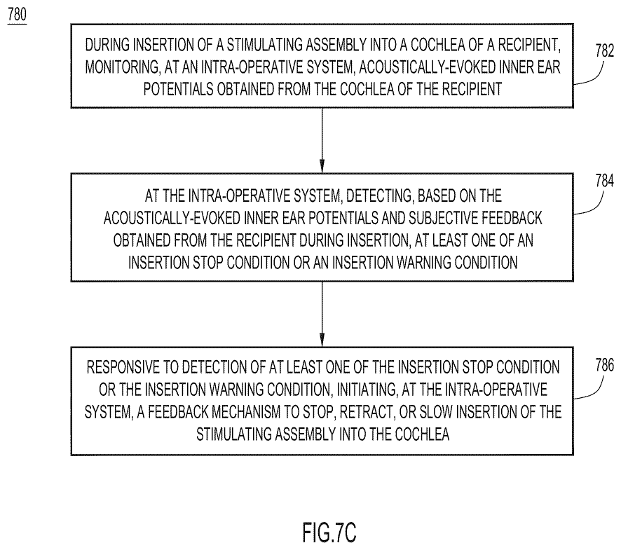

FIG. 7A is a flowchart of a method 700 in accordance with embodiments presented herein. Method 700 begins at operation 702 where, during insertion of a stimulating assembly into a cochlea of a recipient, an intra-operative system monitors acoustically-evoked inner ear potentials obtained from the cochlea of the recipient. At operation 704, the intra-operative system, detects, based on the acoustically-evoked inner ear potentials, an insertion stop condition. At operation 706, responsive to detection of an insertion stop condition, the intra-operative system initiates a feedback mechanism to stop insertion of the stimulating assembly into the cochlea.

FIG. 7B is a flowchart of a method 740 in accordance with embodiments presented herein. Method 740 begins at operation 742 where, during insertion of a stimulating assembly into a cochlea of a recipient, an intra-operative system monitors acoustically-evoked inner ear potentials obtained from the cochlea of the recipient. At operation 744, the intra-operative system, detects, based on the acoustically-evoked inner ear potentials, an insertion warning condition. At operation 746, responsive to detection of an insertion warning condition, the intra-operative system initiates a feedback mechanism to slow insertion of the stimulating assembly into the cochlea, for example.

FIG. 7C is a flowchart of a method 780 in accordance with embodiments presented herein. Method 780 begins at operation 782, where, during insertion of a stimulating assembly into a cochlea of a recipient, an intra-operative system monitors acoustically-evoked inner ear potentials obtained from the cochlea of the recipient. At operation 784, the intra-operative system detects, based on the acoustically-evoked inner ear potentials and subjective feedback obtained from the recipient during insertion, at least one of an insertion stop condition or an insertion warning condition. At operation 786, responsive to detection of at least one of the insertion stop condition or the insertion warning condition, the intra-operative system initiates a feedback mechanism to stop, retract, or slow insertion of the stimulating assembly.

In another variation of the method 780 of FIG. 7C, and in a similar manner as described above with reference to FIG. 4C, since cochlear implant recipients often have some hearing ability in the opposite ear, electrical stimulus could be provided to the first ear 151 of the recipient (in which the stimulating assembly 116 is being inserted) via the stimulating assembly 116 of the cochlear implant 112, and acoustic stimulus could be provided to the second ear 153 of the recipient (the contralateral ear) via the receiver 132 of the hearing aid 131, and the recipient can compare these respective stimuli and advise the surgeon if they are the same or different. For example, in the case of single-sided deafness or residual hearing in the contralateral ear, a tone of the target frequency can be delivered acoustically to the contralateral ear so that the recipient can pitch-match the electrical stimulation delivered to the implanted ear in relation to the acoustic stimulation delivered to the contralateral ear. In the case of residual hearing, this technique can also be used to identify changes to the tonal quality in the implanted ear, where the ECochG tone in the implant ear differs from the contralateral ear. Again, this use of “subjective feedback” from the recipient, in combination with the “objective” measurements, would add confidence regarding whether the target frequency (the target stop point) has been reached. This example embodiment can be particularly advantageous when performing a bilateral implantation, where it can be more important to pitch-match the electrical stimulation in the implanted ear to the acoustic stimulation in the contralateral ear.

In certain embodiments, the techniques described herein can use evoked biological responses to determine relative proximity of electrodes of a stimulating assembly to a target structure in a recipient, such as the modiolus of the cochlea, by estimating and analyzing the relative proximity of multiple (e.g., two or more) electrodes to the target structure. The determined relative modiolar proximity of the electrodes can be used to optimize outcomes for the recipient (e.g., instruct a surgeon or robot to act based on the determined modiolar proximity). The intra-operative system 110 obtains multiple measurements and analyzes the measurements at different electrodes relative to one another to infer perimodiolar position, and thereby infer proximity to the target structure. For example, the techniques presented herein can detect mid electrodes moving away from the modiolus due to over-insertion, and then inform the surgeon (or robotic system) to take a corrective action (e.g., pull back the stimulating assembly) to optimize perimodiolar position with respect to the target structure.

In one specific implementation use case, the middle electrodes (e.g., the electrodes generally between the most apical electrodes and the most basal electrodes) move away from the modiolus when the electrode is over-inserted, which can be corrected by pulling back the electrode (e.g., by hand or with a robot when the surgeon makes use of a robot). As such, using the techniques described between, a surgical system can evaluate the relative modiolar proximity of the electrodes within the electrode array and determine when the stimulating assembly has been inserted (e.g., detect that the middle electrodes are relatively farther away from the modiolus than the apical and/or basal electrodes). As result, the surgical system can notify the surgeon, or instruct a robot, to make direct manipulations to the stimulating assembly.

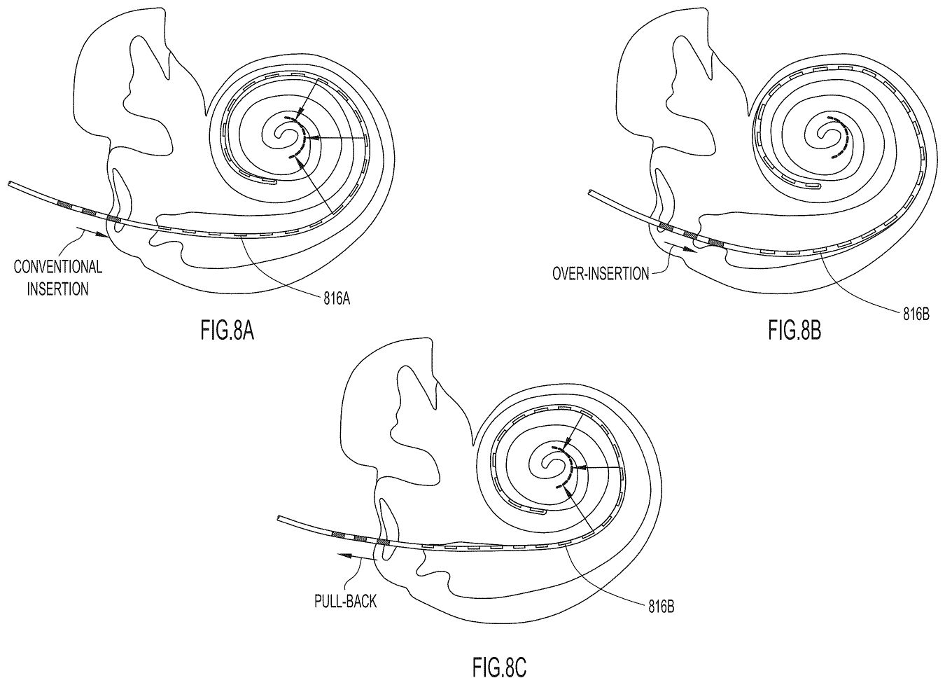

FIGS. 8A, 8B, and 8C schematically illustrate the effect of over-insertion and pull back of a stimulating assembly that can be initiated using the techniques presented herein. In FIGS. 8A, 8B, and 8C, the length of the arrows generally represent the distance between the electrodes and the modiolus. FIG. 8A illustrates a conventional/standard insertion of a stimulating assembly 816A, while FIG. 8B illustrates an “over-insertion” of the stimulating assembly 816B. As noted above in the case of the over-inserted stimulating assembly 816B, the middle electrodes (e.g., the electrodes generally between the most apical electrodes and the most basal electrodes) move away from the modiolus. The positioning of the middle electrodes farther away from the modiolus, relative to the positioning of the apical and/or basal electrodes, can be determined using evoked biological responses captured and analyzed using the techniques presented herein. For example, the SOE spread could show the peak of the SOE function is moving towards the apical end (e.g., that the SOE peak is deviating from a known or desired location, determined based on prior data.), indicating that certain electrodes (e.g., the apical electrodes) are closer to the modiolus than the middle electrodes. FIG. 8C illustrates the over-inserted stimulating assembly 816B of FIG. 8B following a so-called “pull-back” maneuver. As shown, following the pull-back maneuver, the middle electrodes are positioned closer to the modiolus than in the over-inserted position of FIG. 8B.

All above or other measures, individually or in concert, can be used to instruct the surgeon to manipulate the stimulating assembly to obtain the best possible modiolar proximity, such as by pulling back an over-inserted stimulating assembly to get the mid electrode close to the modiolus, inserting the stimulating assembly deeper to assure the basal electrodes get closer to the modiolus, twisting the stimulating assembly to assure the pre-curved stimulating assembly puts itself in the ‘most convenient/least mechanically stressed’ position, etc. As noted, a stimulating assembly can be inserted manually by surgeon or via a partially or fully automated process using a robotic system (e.g., partially or fully automated robotic system). In accordance with certain embodiments, the robotic system can manipulate the stimulating assembly (as indicated above) based on the above measures to achieve a best possible perimodiolar position of the stimulating assembly.

On example use case is that of a recipient with a cochlear implant in a first ear who is undergoing implantation of a second cochlear implant in the contralateral ear. In such a use case, it can be desirable to avoid loss of the residual hearing in the second ear and/or match pitch perception in the second ear to the first ear. In this instance, a surgeon could use a pitch match between the two electrodes (electrical stimulus) to decide the stop condition instead of, or as well as, an acoustic input.

As noted above, presented herein are techniques for monitoring the insertion of an intra-cochlear stimulating assembly for the occurrence of one or more insertion stop or warning conditions. Also as noted, the insertion monitoring is based on objectively measured inner ear responses/potentials, such as acoustically-evoked potentials. The intra-operative insertion monitoring system and the cochlear implant system described herein can provide a continuous feedback system that enables an iterative approach to be taken with respect to insertion of the stimulating assembly and reaching the intended target frequency (target stop point). In addition to indicating that the target position has been reached, the intra-operative system can also indicate that the target has been over-shot (e.g., indicates to pull-back/retract the stimulating assembly) or that the stimulating assembly is nearing the target (i.e., indicates to slow-down insertion of the stimulating assembly). In certain examples, the insertion monitoring based on objective measurements can also be combined with “subjective feedback” obtained from the recipient during insertion (e.g., to further improve accuracy of the insertion on an individual patient basis). As a result, a feature of the techniques presented herein is an improved ability to preserve the recipient's post-operative residual hearing through the provision of dynamic and static insertion metrics.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments may be combined with another in any of a number of different manners.

What is claimed is:

1. A method, comprising:
obtaining intra-operative acoustically-evoked inner ear potentials from a cochlea of a recipient of a cochlear implant, wherein the intra-operative acoustically-evoked inner ear potentials are obtained via at least one stimulating electrode of a stimulating assembly of the cochlear implant;
determining, from the intra-operative acoustically-evoked inner ear potentials, a tonotopic frequency of the cochlea at a position of the at least one stimulating electrode within the cochlea; and
aligning the stimulating assembly with a tonotopy of the cochlea by matching a frequency associated with the at least one stimulating electrode to the tonotopic frequency of the cochlea at the position of the at least one stimulating electrode within the cochlea.

2. The method of claim 1, wherein the method comprises:
advancing the stimulating assembly within the cochlea to change a tonotopic position of the at least one stimulating electrode within the cochlea.

3. The method of claim 2, wherein the method comprises:
advancing the stimulating assembly to a tonotopic position within the cochlea that matches a surgical plan for the recipient.

4. The method of claim 1, wherein the method comprises:
matching the frequency associated with a plurality of stimulating electrodes of the stimulating assembly to the tonotopic frequency of the cochlea at the position of the plurality of stimulating electrodes within the cochlea.

5. The method of claim 1, wherein the method comprises:
a surgical robot applying closed-loop control, with the intra-operative acoustically-evoked inner ear potentials as feedback, to align the stimulating assembly with the tonotopy of the cochlea.

6. The method of claim 1, wherein the method comprises:
determining, from the intra-operative acoustically-evoked inner ear potentials, a proximity of the stimulating assembly to a target position within the cochlea; and
controlling insertion of the stimulating assembly based on the proximity of the stimulating assembly to the target position.

7. The method of claim 6, wherein controlling insertion of the stimulating assembly based on the proximity of the stimulating assembly to the target position comprises:
controlling an insertion speed of the stimulating assembly based on the proximity of the stimulating assembly to the target position.

8. A method, comprising:
matching a frequency associated with at least one stimulating electrode of a cochlear implant to a tonotopic frequency of a cochlea of a recipient at a position of the at least one stimulating electrode based on intra-operative acoustically-evoked inner ear potentials obtained by the cochlear implant during insertion of a stimulating assembly into the cochlea.

9. The method of claim 8, comprising:
iteratively obtaining, during insertion of the stimulating assembly into the cochlea of the recipient, the intra-operative acoustically-evoked inner ear potentials of the cochlea via the at least one stimulating electrode; and
iteratively determining, during insertion of the stimulating assembly into the cochlea of the recipient and based on the intra-operative acoustically-evoked inner ear potentials, a tonotopic region of the cochlea at which the at least one stimulating electrode is positioned.

10. The method of claim 9, wherein matching a frequency associated with at least one stimulating electrode of a cochlear implant to a tonotopic frequency of a recipient's cochlea at the position of the at least one stimulating electrode comprises:
determining when the at least one stimulating electrode has reached a target tonotopic location of the cochlea.

11. The method of claim 10, further comprising:
responsive to determining when the at least one stimulating electrode has reached the target tonotopic location of the cochlea, initiating a feedback mechanism to stop insertion of the stimulating assembly into the cochlea.

12. The method of claim 11, wherein initiating the feedback mechanism comprises at least one of stopping automated insertion of the stimulating assembly or generating a stop notification for a surgeon to stop insertion of the stimulating assembly.

13. The method of claim 8, wherein the method comprises:
determining, from the intra-operative acoustically-evoked inner ear potentials, a proximity of the at least one stimulating electrode to a target position within the cochlea; and
controlling insertion of the stimulating assembly based on the proximity of the stimulating assembly to the target position.

14. The method of claim 13, wherein controlling insertion of the stimulating assembly based on the proximity of the stimulating assembly to the target position comprises:
controlling an insertion speed of the stimulating assembly based on the proximity of the at least one stimulating electrode to the target position.

15. A method, comprising:
obtaining acoustically-evoked inner ear potentials from a recipient undergoing cochlear implant surgery to insert a stimulating assembly into a cochlea of the recipient, and
detecting a change in the acoustically-evoked inner ear potentials indicative of a change in mechanical impedance of a basilar membrane of the recipient.

16. The method of claim 15, further comprising:
responsive to detection of the change in the acoustically-evoked inner ear potentials indicative of a change in mechanical impedance of a basilar membrane of the recipient, initiating a feedback mechanism.

17. The method of claim 16, wherein initiating the feedback mechanism comprises at least one of stopping automated insertion of the stimulating assembly or generating a stop notification for a surgeon to stop insertion of the stimulating assembly.

18. The method of claim 16, wherein initiating the feedback mechanism comprises at least one of initiating automated retraction of the stimulating assembly or generating a retract notification for a surgeon to at least partially retract the stimulating assembly.

19. The method of claim 15, wherein the acoustically-evoked inner ear potentials are obtained via at least one stimulating electrode of the stimulating assembly during insertion of the stimulating assembly into the cochlea, and wherein the method further comprises:
determining, based on the mechanical impedance of a basilar membrane of the recipient, that the stimulating assembly has physically contacted the basilar membrane.

20. The method of claim 15, wherein the acoustically-evoked inner ear potentials are obtained via at least one stimulating electrode of the stimulating assembly during insertion of the stimulating assembly into the cochlea, and wherein the method further comprises:
determining, based on the mechanical impedance of a basilar membrane of the recipient, that the stimulating assembly is in proximity to, but has not yet physically contacted, the basilar membrane.
